(19) United States
(12) Patent Application Publication
(10) Pub. No.: US 2015/0272438 A1
(43) Pub. Date: Oct. 1, 2015

(54) IMAGING RETINAL INTRINSIC OPTICAL SIGNALS

(71) Applicant: THE UAB RESEARCH FOUNDATION, Birmingham, AL (US)

(72) Inventors: Xincheng Yao, Hoover, AL (US); Qiuxiang Zhang, Birmingham, AL (US); Rongwen Lu, Birmingham, AL (US)

(73) Assignee: The UAB RESEARCH FOUNDATION, Birmingham, AL (US)

(21) Appl. No.: 14/438,425

(22) PCT Filed: Oct. 24, 2013
(86) PCT No.: PCT/US2013/066545

§ 371 Date: Apr. 24, 2015

Related U.S. Application Data
(60) Provisional application No. 61/717,679, filed on Oct. 24, 2012.

(51) Int. Cl. A61B 3/14; A61B 3/10; A61B 3/00

(52) U.S. Cl. CPC A61B 3/14; A61B 3/0008; A61B 3/102

(57) ABSTRACT

Disclosed are various embodiments for imaging retinal intrinsic optical signals (IOS) in vivo. According to various embodiments, imaging retinal intrinsic optical signals (IOS) may comprise illuminating a host retina with near infrared light (NIR) during a test period, wherein the host retina is continuously illuminated by the NIR light during the test period. Sequentially a host retina may be stimulated with a timed bursts of visible light during the test period. A series of images of the retina may be recorded with a line-scan CCD camera and the images may be processed to produce images of intrinsic optical signals (IOS) from retinal photoreceptor cells identified in the images.

### CROSS-REFERENCE TO RELATED APPLICATIONS

This application claims the benefit of and priority to U.S. Provisional Application Ser. No. 61/717,679, filed Oct. 24, 2012, and entitled “METHODS AND APPARATUS FOR IMAGING RETINAL INTRINSIC OPTICAL SIGNALS” which is incorporated by reference herein in its entirety.

### FEDERAL SPONSORSHIP

This invention was made with Government support under Contract/Grant No. CBET-1055889, awarded by the U.S. National Science Foundation, and under Contract/Grant No. R21EB012264, awarded by the U.S. National Institutes of Health. The Government has certain rights in this invention.

### BACKGROUND

It is well established that many eye diseases involve pathological changes of photoreceptors and/or their support system, including different forms of retinitis pigmentosa (RP) and age-related macular degeneration (AMD), a highly prevalent outer retinal disease. Age-related macular degeneration (AMD) is the leading cause of severe vision loss and legal blindness. In the U.S. alone, more than 10 million people are estimated to have early AMD. For example, 1.75 million patients are currently suffering visual impairment due to late AMD.

To prevent or slow the progress of vision loss associated with outer retinal disease, early detection and reliable assessment of medical interventions, including morphological examinations, are key elements. The application of adaptive optics (AO) and optical coherence tomography (OCT) has enabled retinal fundus imaging with cellular resolution. However, disease-associated morphological and functional changes, if independently measured, are not always correlated directly in time course and spatial location. Therefore, a combined assessment of retinal function and structure is essential.

Psychophysical methods that access outer retinal function, such as visual acuity (VA) testing, are practical in clinical applications; however, VA testing involves extensive higher order cortical processing. Therefore, VA testing does not provide information on retinal function exclusively and lacks sensitivity for early detection of outer retinal diseases, such as AMD. Electroretinography (ERG) methods, including full-field ERG, focal ERG, multifocal ERG, etc., have been established for objective examination of retinal function. However, the spatial resolution of ERG may not be high enough to provide direct comparison of localized morphological and functional changes in the retina.

### SUMMARY

According to various embodiments of the present disclosure, disclosed is a method for imaging retinal intrinsic optical signals (IOS) in vivo comprising: illuminating a host retina with near infrared light (NIR) during a test period, wherein the host retina is continuously illuminated by the NIR light during the test period; sequentially stimulating a host retina with a timed bursts of visible light during the test period; recording a series of images of the retina with a line-scan CCD camera, wherein images are recorded both before, after, and during stimulus of the retina with the visible light; and processing the images to produce images of intrinsic optical signals (IOS) from retinal photoreceptor cells identified in the images.

According to various embodiments of the present disclosure, disclosed is an imaging system for in vivo retinal imaging of a host retina comprising: at least one computing device; and a line-scan confocal ophthalmoscope comprising: a linear CCD camera; a near infrared (NIR) light source; a visible light source; a scanning mirror; an adjustable mechanical slit disposed between the visible light source and the host retina; and a near infrared (NIR) filter disposed between the visible light source and the camera to block visible stimulus light; and an application executable by the at least one computing device, the application comprising: logic that obtains images recorded by the camera; logic that stores the recorded images in a storage device accessible to the at least one computing device; and logic that processes the images to produce images of intrinsic optical signals (IOS) from retinal photoreceptor cells.

According to various embodiments of the present disclosure, disclosed is an imaging system for in vivo retinal imaging of a host retina, comprising: a line-scan confocal ophthalmoscope comprising: a linear CCD camera; a near infrared (NIR) light source; a visible light source; a scanning mirror; an adjustable mechanical slit disposed between the visible light source and the host retina; and a near infrared (NIR) filter disposed between the visible light source and the camera to block visible stimulus light, wherein, the system is capable of processing the images recorded by the camera to produce images of intrinsic optical signals (IOS) from retinal photoreceptor cells.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIGS. 1A-B are schematic diagrams a line-scan confocal ophthalmoscope for reflected light IOS imaging according to various embodiments of the present disclosure.

FIG. 2 is an example of imaging and data captured utilizing a northern leopard frog according to various embodiments of the present disclosure.

FIG. 3 is an image depicting a confocal image of a frog retina and a plurality of spatial IOS image sequences according to various embodiments of the present disclosure.

FIG. 4 is an image describing comparative IOS and ERG analysis according to various embodiments of the present disclosure.

FIG. 5 is an image describing stimulus flashes presented at predefined intervals according to various embodiments of the present disclosure.

FIG. 6 is a near infrared (NIR) image of frog photoreceptors according to various embodiments of the present disclosure.

FIG. 7 is an image depicting oblique stimulus-evoked photoreceptor displacements according to various embodiments of the present disclosure.

FIGS. 8A-B are images depicting comparisons of rod and cone displacements according to various embodiments of the present disclosure.

FIGS. 9A-D are images depicting transient photoreceptor displacement correlated with circular stimulation according to various embodiments of the present disclosure.

FIGS. 10A-B are drawings of an in vivo image (500×400 pixels) of a frog retina according to various embodiments of the present disclosure.

FIGS. 11A-C are drawings of representative data of a spatial IOS image sequence according to various embodiments of the present disclosure.

FIGS. 12A-C are schematic diagrams of stimulation patterns according to various embodiments of the present disclosure.

FIGS. 13A-E are images depicting oblique stimulus-evoked photoreceptor displacements according to various embodiments of the present disclosure.

FIGS. 14A-G are images depicting photoreceptor displacements and intrinsic optical signal (IOS) responses stimulated by circular stimulus (in transverse plane) according to various embodiments of the present disclosure.

FIGS. 15A-C are images depicting stimulus-evoked photoreceptor displacements at the mouse retina according to various embodiments of the present disclosure.

FIGS. 16A-B are schematic diagrams of a time domain LS-OCT according to various embodiments of the present disclosure.

FIGS. 17A-B are images showing LS-OCT pictures of a frog eyecup obtained according to various embodiments of the present disclosure.

FIG. 18 is a flowchart illustrating one example of performing in vivo imaging of intrinsic optical signals from retinas according to various embodiments of the present disclosure.

FIG. 19 is a flowchart illustrating one example of functionality implemented as portions of an imaging application executed in a computing device or a computing environment according to various embodiments of the present disclosure.

FIG. 20 is a schematic block diagram that provides one example illustration of a computing environment employed to conduct in vivo imaging of intrinsic optical signals from retinas.

### DETAILED DESCRIPTION

The present disclosure relates to in vivo imaging of intrinsic optical signals from retinas. The present disclosure provides discussion of imaging, mapping, and detection of retinal injury and/or dysfunction, such as those associated with certain retinal conditions, including various outer retinal diseases. The present disclosure also describes detecting and/or diagnosing retinal conditions using various methods and systems described within the present disclosure. The present disclosure includes involving orientation-dependent stimulation to evaluate rod photoreceptor physiology and function.

Further, the present disclosure describes the physiological mechanism of stimulus-evoked fast intrinsic optical signals (IOSs) recorded in dynamic confocal imaging of the retina, and demonstrates in vivo confocal-IOS mapping of localized retinal dysfunctions. The present disclosure also demonstrates an orientation-dependent IOS biomarker for selective functional mapping of rod photoreceptor physiology.

As described in greater detail in the examples below, a rapid line-scan confocal ophthalmoscope may be employed to achieve in vivo confocal-IOS imaging of retinas such as human retinas, frog retinas (e.g., Rana pipiens retinas), and/or mouse retinas (e.g., Mus musculus retinas), at a cellular resolution. According to one embodiment, in order to investigate the physiological mechanism of confocal-IOS, comparative IOS and electroretinography (ERG) measurements may be conducted using normal frog eyes activated by variable intensity stimuli. A dynamic spatiotemporal filtering algorithm may be employed to reject a contamination of hemodynamic changes in fast IOS recording. Laser-injured frog eyes may be employed to test the potential of confocal-IOS mapping of localized retinal dysfunctions.

Comparative IOS and ERG experiments described below revealed a close correlation between the confocal-IOS and retinal ERG, particularly the ERG a-wave which has been widely used to evaluate photoreceptor function. IOS imaging of laser-injured frog eyes indicates that the confocal-IOS can unambiguously detect localized (30 μm) functional lesions in the retina before a morphological abnormality is detectable. The confocal-IOS predominantly results from retinal photoreceptors, and can be used to map localized photoreceptor lesion in laser-injured frog eyes. These confocal-IOS imaging techniques can provide applications in early detection of age-related macular degeneration, retinitis pigmentosa, and/or other retinal diseases that can cause pathological changes in the photoreceptors.

Stimulus-evoked fast intrinsic optical signals (IOSs) are a promising alternative to ERG for objective measurement of retinal function that also provides improved spatial resolution. Ex vivo IOS identification of localized retinal dysfunction may be demonstrated in an inherited photoreceptor degeneration model. Because functional IOS images are constructed through spatiotemporal processing of pre- and post-stimulus images, concurrent structural and functional measurements can be naturally achieved using a single optical instrument. Conventional fundus cameras may be employed to detect IOSs from anesthetized cats and monkeys and awake humans. Given limited axial resolution, fundus IOS imaging does not exclusively reflect retinal neural function due to complex contaminations of other ocular tissues. In principle, adaptive optics and optical coherence tomography (OCT) imagers may provide cellular resolution. However, a signal source and mechanism of these imaging modalities are not well established, and functional mapping of fast IOSs that have time courses comparable to retinal electrophysiological kinetics is still challenging.

According to various embodiments, a line-scan confocal microscope to may be employed to achieve fast IOS imaging at high-spatial (μm) and high-temporal (ms) resolutions. Rapid in vivo confocal-IOS imaging has revealed a transient optical response with a time course comparable to ERG. Embodiments described below report comparative confocal-IOS imaging and retinal ERG recording for investigating the physiological mechanism of confocal-IOS33-35, and demonstrate confocal-IOS identification of localized acute retinal lesions in an animal model, i.e., laser-injured frog eyes.

Embodiments of methods and systems of the present disclosure are described briefly below. Specifics of the methods and systems of the present disclosure will be described in greater detail in following examples. Briefly described, embodiments of the present disclosure include methods of imaging retinal intrinsic optical signals (IOS) in vivo. In embodiments, methods include illuminating a host retina with near infrared light during a test period, wherein the host retina is continuously illuminated by a near infrared (NIR) light during the test period; sequentially stimulating a host retina with a timed bursts of visible light during the test period; recording a series of images of the retina with a line-scan CCD camera, wherein images are recorded before, after, and/or during stimulus of the retina with the visible light; and processing the images to produce images of intrinsic optical signals (IOS) from retinal photoreceptor cells. In various embodiments, the visible light stimulus may be a visible green light or a white light. According to various embodiments, the light is specifically directed at portions of the retina with an adjustable mechanical slit disposed between the visible light source and the host retina to focus the light stimulus on a specific area of the retina. In embodiments, the NIR light can be about 100 μW to 1200 μW or, in some embodiments, about 600 μW. In embodiments the visible light is filtered from the camera with an NIR filter. In embodiments the bursts of visible light are timed at specific intervals which may be synchronized with the timing of the image acquisition by the camera. The images may be recorded at specified intervals for specified amounts of time before, during, and/or after delivery of the stimulus. In embodiments images are recorded for a period of time beginning about 100 ms to 800 ms or, in some embodiments, about 400 ms before the stimulus and continuing until about 100 ms to 2000 ms or, in some embodiments, 800 ms after the stimulus at intervals of about 10 to 1000 frames/s or, in some embodiments, 100 frames/s. The images are recorded by the camera and processed to produce IOS images of the host retina. In embodiments blood flow dynamics are filtered from the image to separate IOS from optical changes induced by blood flow from ocular blood vessels. This can be done by programs using algorithms, such as those described below, for accounting for and filtering changes attributable to blood flow dynamics. The images can be processed to show IOS images from photoreceptors, such that the absence of IOS signals or reduced signal in an area of an image indicates the location of photoreceptor damage. Also, images of control retinas can be compared to images of inured retina, where differences in the images can indicate the location of injured photoreceptors.

In some embodiments the visible light is directed at an oblique angle of about 15° to 60° or, in some embodiments, about 30° relative to the normal axis of the retinal surface visible light is stimulated a circular pattern on the retina. This pattern of stimulus allows imaging of photoreceptor rods, by imaging IOS produced by transient phototropic change of retinal rods, as described in greater detail in below.

Embodiments of the present disclosure, briefly described, also include imaging systems for in vivo retinal imaging of a host retina. Such embodiments may comprise a line-scan confocal ophthalmoscope including a camera (e.g., a linear CCD camera), a near infrared (NIR) light source, a visible light source, a scanning mirror, an adjustable mechanical slit disposed between the visible light source and the host retina, and a near infrared (NIR) filter disposed between the visible light source and the camera to block visible stimulus light, where the system is capable of processing the images recorded by the camera to produce images of intrinsic optical signals (IOS) from retinal photoreceptor cells. In embodiments the system also includes at least one computing device for processing the images to produce the IOS images. In such embodiments, the system includes at least one application executable by the computing device, where the application includes logic that obtains images recorded by the camera, logic that stores the recorded images in a storage device accessible to the at least one computing device, and logic that processes the images to produce images of IOS from retinal photoreceptor cells. In embodiments, the application also includes logic that filters blood flow dynamics to separate IOS from optical changes induced by blood flow from ocular blood vessels.

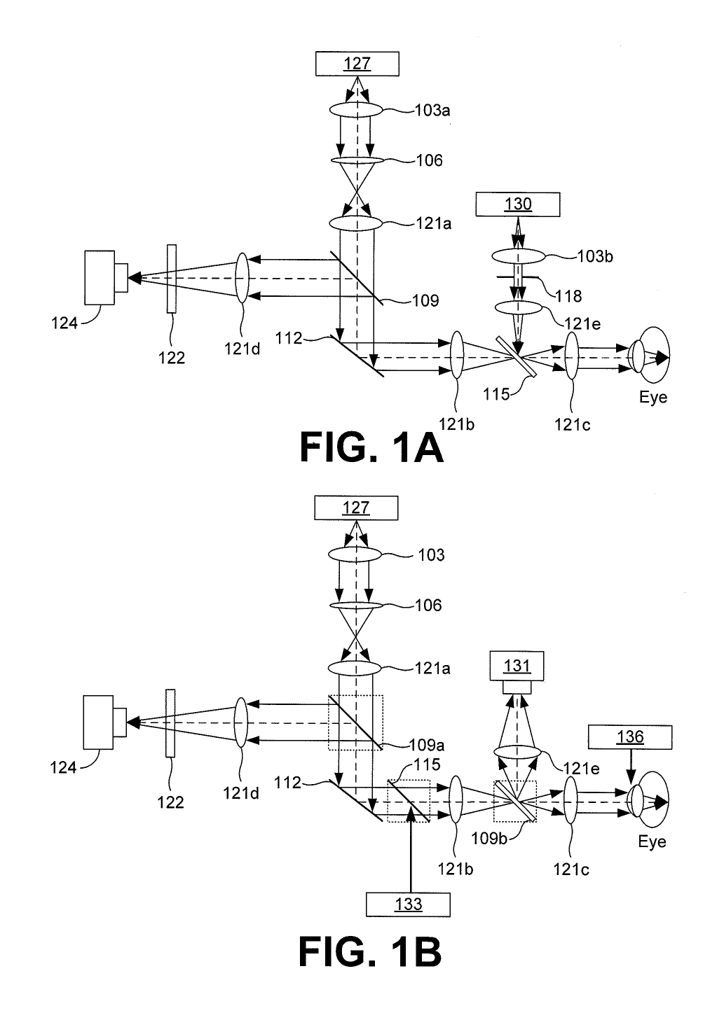

With respect to FIG. 1A, shown is a schematic diagram of a non-limiting example of a line-scan confocal ophthalmoscope for confocal-IOS imaging. In the non-limiting example of FIG. 1A, the scan-scan confocal ophthalmoscope may comprise one or more collimators (CO) 103a and 103b; a cylindrical lens (CL) 106; a beam splitter (BS) 109; a scanning mirror (SM) 112; a dichroic mirror (DM) 115; one or more mechanical slits (MS) 118; one or more optical lenses (LX) 121a, 121b, 121c, 121d, and 121e; an NIR filter 122; and/or other components. According to various embodiments, the imaging system may employ a linear CCD camera 124 such as an EV71YEM2CL1014-BA0 camera (E2V, New York, USA). The CCD camera 124 may be equipped with a camera link interface that is configured to facilitate system control and data synchronization. The line-scan confocal imaging system may comprise one or more light sources. For example, a near infrared (NIR) light source 127 may be employed for IOS recording and a visible green light source 130 may be employed for retinal stimulation. According to various embodiments, the NIR light source 127 may comprise a superluminescent laser diode (SLD) such as a SLD-35-HP diode (Superlum, Co. Cork, Ireland) with a center wavelength of 830 nm.

In an embodiment of the present disclosure, a single-mode fiber coupled 532-nm DPSS laser module, such as a FC-532-020-SM-APC-1-1-ST (RGBLase LLC, California, USA), may be utilized to produce visible light for stimulating or injuring the retina locally. For example, the laser module may be configured to provide adjustable output power from 0 to 20 mW at the fiber end. A mechanical slit 118, such as the VA100 (Thorlabs, New Jersey, USA), may be configured to be placed behind the collimated green stimulus light to produce a rectangle pattern and provide precise adjustment of stimulus width.

A software application, configured to be executed in a computing device, may be configured to provide a real-time image display, high-speed image acquisition, and signal synchronization. Before each IOS recording, stimulus timing and location in the field of view may be tested for repeatability and accuracy. During each testing, a retina subject to the recording may be continuously illuminated by the NIR light source 127 at or around ˜600 μW. As a non-limiting example, for each IOS recording testing, 400 ms pre-stimulus and 800 ms after-stimulus images may be recorded at the speed of 100 frames/s with frame size of 350×100 pixels (˜300 μm×85 μm at the retina). Exposure time of the line-scan CCD camera 124 may be configured at about 71 μs or, in some embodiments, about 71.4286 μs and scanning speed of the mirror may be configured at about 50 to 150 Hz or, in some embodiments, about 100 Hz.

Electroretinography (ERG) may be recorded by placing differential electrodes on two eyes of a subject, such as a human, frog, or mouse. The ERG signal may be amplified with a physiological amplifier, such as the DAM 50 (World Precision Instruments, Florida, USA), which is equipped with a band-pass (0.1 Hz to 10 kHz) filter. The pre-amplified ERG may be digitized using, for example, a 16-bit DAQ card such as the NI PCIe-6351 (National Instruments®, Texas, USA) with a resolution of 1.6 mV. The pre-amplified ERG may be sent to a computing device for averaging, display, and storage, as may be appreciated.

With respect to FIG. 1B, shown is a schematic diagram of another embodiment of a line-scan confocal ophthalmoscope for confocal-IOS imaging. In the non-limiting example of FIG. 1B, the scan-scan confocal ophthalmoscope may comprise a collimator (CO) 103; a cylindrical lens (CL) 106; one or more beam splitters (BS) 109a and 109b; a scanning mirror (SM) 112; a dichroic mirror (DM) 115; one or more optical lenses (LX) 121a, 121b, 121c, and 121d; an NIR filter 122; a camera 131, such as a full-field CCD camera; a light source (e.g., a green LED) 133; a near infrared light source (e.g., NIR LED) 136; and/or other components. According to various embodiments, the imaging system may employ a linear CCD camera 124 such as an EV71YEM2CL1014-BA0 camera (E2V®, New York, USA). The camera 131 (e.g., full-field CCD camera) may be equipped with a camera link interface that is configured to facilitate system control and data synchronization. The line-scan confocal imaging system may comprise one or more light sources, such as light source 133 and NIR light source 136. For example, the near infrared (NIR) light source 136 may be employed for IOS recording and a visible green light source 133 may be employed for retinal stimulation. According to various embodiments, the NIR light source 136 may comprise a superluminescent laser diode (SLD) such as a SLD-35-HP diode (Superlum®, Co. Cork, Ireland) with a center wavelength of 830 nm.

In the schematic diagram of the line-scan confocal ophthalmoscope is depicted in FIG. 1B, a fast linear CCD camera 124, such as a SG-11-01k80-00R (DALSA), with a pixel size of 14 μm×14 μm and pixel a sampling rate up to 80 MHz, may be employed to achieve high-speed and high-resolution imaging. A line-scan confocal microscope may be modified to an animal ophthalmoscope for in vivo imaging of the retina. In this embodiment, a NIR (center wavelength: 830 nm; bandwidth: 60 nm) superluminescent laser diode (SLD), such as the SLD-35-HP (Superlum®, Co. Cork, Ireland) may be used for IOS imaging, and a green light-emitting diode (LED) 133 may be used for retinal stimulation. Moreover, a NIR LED 136 may be placed beside or near the eye to provide oblique illumination of the pupil, and a full-field CCD camera 130 may be used to monitor the pupil to allow easy alignment of the NIR SLD light 127 for IOS recording. The cylindrical lens (CL) 106 condensed the NIR recording light into one dimension to produce a focused line illumination, which was conjugated with the linear CCD camera 124. Lateral and axial resolutions of the system are theoretically estimated ˜1 μm and ˜10 μm, respectively.

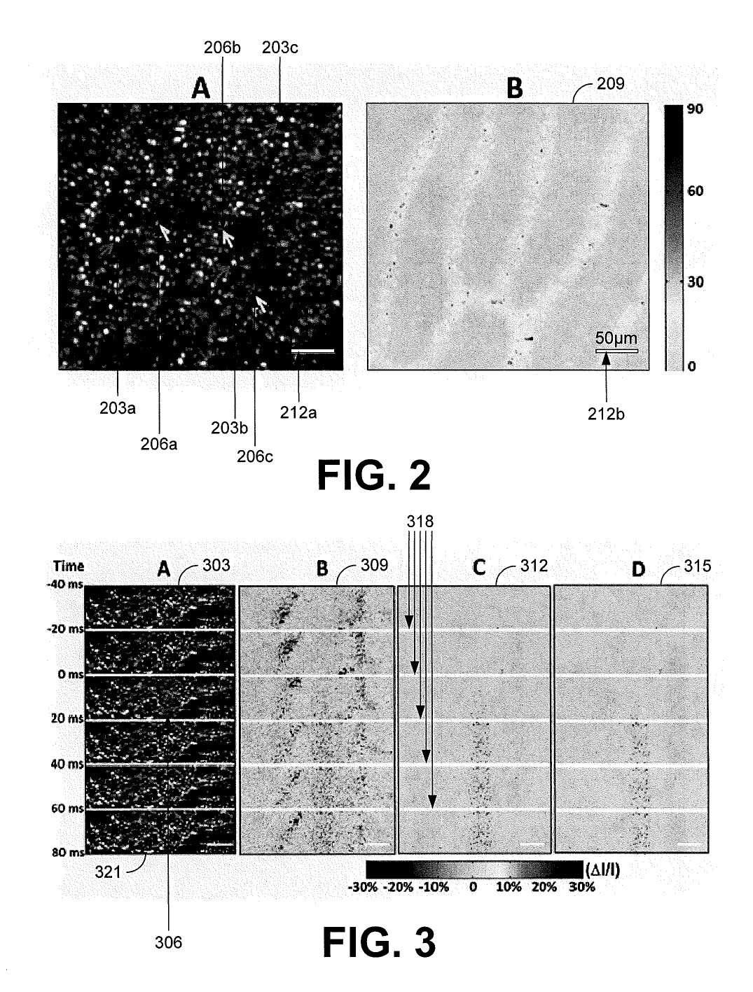

Moving on to FIG. 2, shown is an example of imaging and data captured utilizing a northern leopard frog (Rana pipiens). In the non-limiting example of FIG. 2, the northern leopard frog may be used to take advantage of the high-quality optics of the ocular lens and the large size of the retinal photoreceptors (cone, 3 μm; rod, 6 μm). Together, these characteristics may resolve individual photoreceptor cells 203a, 203b, and 203c, as well as blood vessels 206a, 206b, and 206c in vivo. The experimental procedure used in generating the imaging and data of depicted in FIG. 2, as well as other portions of the present disclosure, was approved by the Institutional Animal Care and Use Committee of the University of Alabama at Birmingham and carried out in accordance with the guidelines of the ARVO Statement for the Use of Animals in Ophthalmic and Vision Research. Frogs were dark adapted for at least 2 hours prior to functional IOS imaging. The frog was then anesthetized by immersion in tricaine methanesulfonate solution (TMS, MS-222; 500 mg/liter). Pupils were fully dilated with topical atropine (0.5%) and phenylephrine (2.5%). After confirmation of the anesthesia, the frog was placed in a custom-built holder for IOS imaging. The holder provided five degrees of freedom to facilitate adjustment of body orientation and retinal area for IOS imaging.

As shown in FIG. 2, ocular blood vessels 206a, 206b, and 206c can superimpose on photoreceptor cells 203a, 203b, and 203c and hemodynamic changes inherent to rapid blood flow may contribute to fast (OS recording. Retinal blood vessels can be mapped based on dynamic optical changes correlated with blood flow. A stimulus-evoked fast IOS in retinal photoreceptors may be separated from a blood flow-induced optical change. Key procedures of the dynamic spatiotemporal filtering are summarized as follows:

To calculate the mean Ī(x, y) of each pixel in the pre-stimulus baseline recording (n frames), eq. 1 may be employed:

$$\overline{I} ( x , y ) = \frac{1}{n} {\sum }_{j = 1}^{j = n} {I}_{{t}_{j}} ( x , y ) . \qquad ( eq . 1 )$$

To calculate the standard deviation σ(x, y) of each pixel in the pre-stimulus baseline recording (n frames), eq. 2 may be employed to conduct spatiotemporal filtering of potential noises:

$$σ ( x , y ) = \sqrt{\frac{1}{n} {\sum }_{j = 1}^{j = n} {[ {I}_{{t}_{j}} ( x , y ) - \overline{I} ( x , y ) ]}^{2}} . \qquad ( eq . 2 )$$

Because blood flow changes dynamically, the variability of light intensity at the blood vessels in temporal is much larger than it is at the blood-free area, i.e., before the stimulus, the temporal σ(x, y) of blood flow is much larger than that of photoreceptors. Upon stimulation, blood flow may increase, but within a short recording time (˜1 s), hemodynamic change is much slower than stimulus-evoked photoreceptor activation. Therefore, the temporal change of blood flow σ(x, y) may be described as insignificant compared with the fast IOSs from the photoreceptors. To reject noise attributable to blood flow, values three standard deviations above or below the mean at each pixel may be employed as a filtering criterion. This filter (3-σ) permits the plotting of the vasculature profile 209 as shown in FIG. 2.

In other words, the pixel change will be assumed to reflect noise, if

I̅(x,y)−3σ(x,y)<Iₜ_{ᵢ}(x,y)< I̅(x,y)+3σ(x,y) (eq. 3).

Therefore, a high threshold is used to define stimulus-evoked IOS in the retinal area superimposed by blood vessels. The signals at pixel (x, y) with light intensity greater than the mean above three standard deviations are positive and less than the mean below three standard deviations are negative. IOS images with pixels that fall into the noise range are forced to be zero and only positive or negative IOSs are left. Therefore, after dynamic spatiotemporal filtering, most hemodynamic-driven optical signals (can be rejected, as will be discussed in greater detail below with respect to FIG. 3. In portion (b) of FIG. 2, a 3-σ map of the pre-stimulus images shows a blood vessel pattern, wherein scale bars 212a and 212b represent 50 μm.

In FIG. 3, shown is a confocal image of a frog retina 303, wherein each illustrated frame was the average over 20 ms. Epochs of 40 ms (pre-stimulus) and 80 ms (post-stimulus) are shown. The rectangle 306 depicted in the third frame of the confocal image of a frog retina 303 indicates the size and location of the stimulus pattern relative to the region of interest in the retina. FIG. 3 further depicts the spatial IOS image sequence 309 before filtering blood dynamics, the spatial IOS image sequence 312 after filtering blood dynamics, and the IOS strength distribution image sequence 315 after filtering blood dynamics. A scale bar 318 represents, for example, 50 μm.

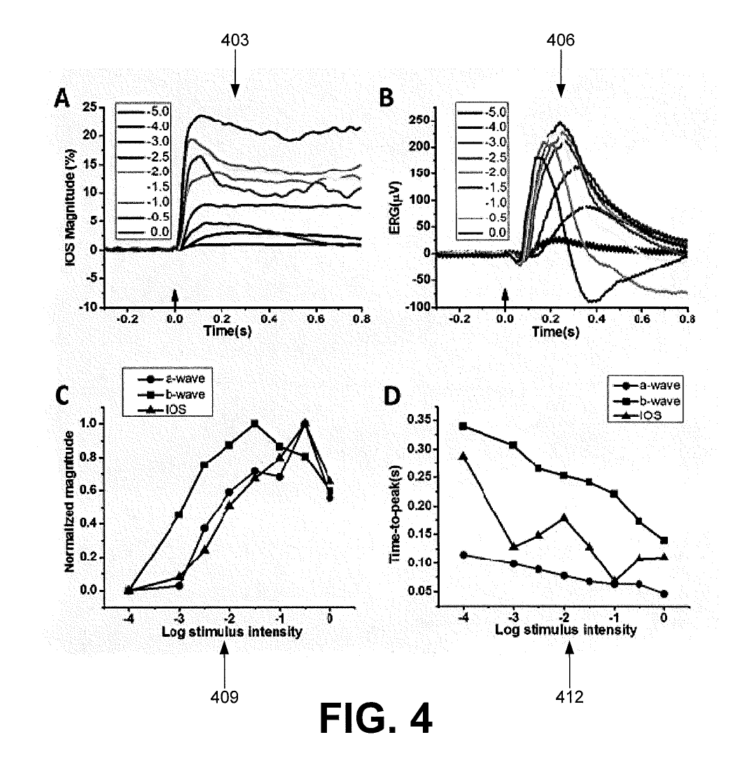

A rectangular stimulus bar 321 with 30-μm width and a 20-ms duration may be used to depict localized retinal stimulation. In the non-limiting example of FIG. 3, estimated maximum stimulus flash intensity was set to 3.5×105 photons/μm2/ms (7×106 photons/μm2 for 20 ms) at the retina. Neutral density filters may be employed to adjust light intensity for retinal stimulation. The IOS and ERG were recorded over a 5.0 log unit range in 9 steps, namely, −5.0, −4.0, −3.0, −2.5, −2.0, −1.5, −1.0, −0.5, and 0.0. Stimulus flashes were presented at 2-minute intervals, as described below with respect to FIG. 4. IOS and ERG recordings were performed consecutively in the same frog eye.

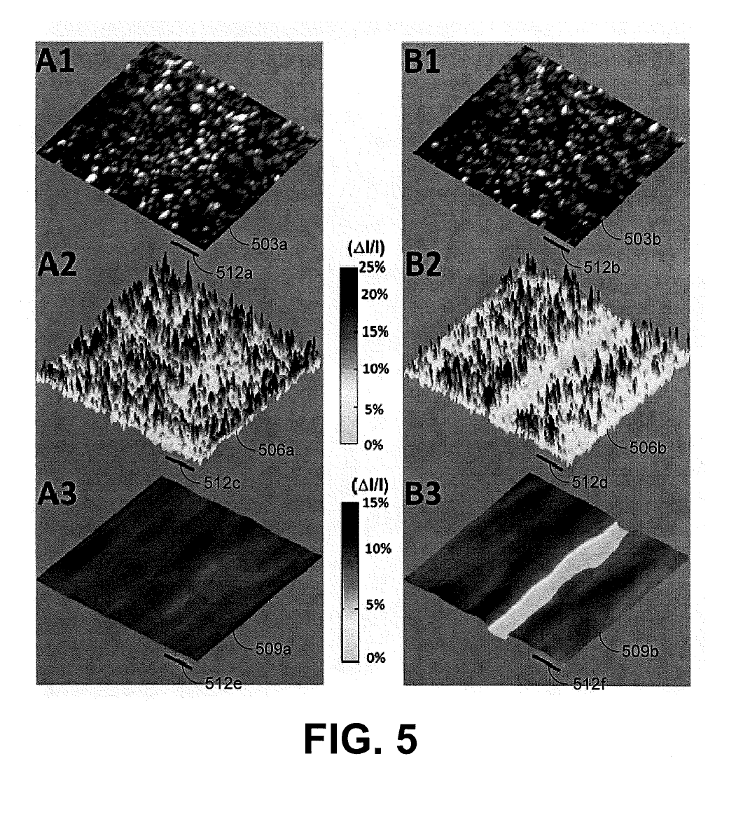

Both normal and laser-injured frogs were used in this study. To produce a localized retina laser-injury, a 30-μm width green laser light bar with output power of 1 mW at the retina surface was continuously delivered into the retina for 30 s. Thirty minutes after local damage was induced, a full-field stimulus, described below with respect to FIG. 5, was applied to injured and non-injured area to obtain the retinal response pattern.

Each illustrated frame in FIG. 3 is the average of two raw/IOS images obtained during a 20-ms epoch. Additionally, 40-ms pre-stimulus and 80-ms post-stimulus recordings are shown. The spatial IOS image sequence 309 was observed after a rectangular stimulus was delivered, whereas the blood vessels showed persistent optical changes. After setting the pixels falling within the range defined by eq. 3 to zero, most of the rapid blood flow activities were excluded from stimulus-evoked retinal responses. With a clean background, the stimulus activated (OS pattern can be visualized clearly in spatial IOS image sequence 312. Both positive and negative signals may be observed almost immediately after retinal stimulation. Spatial IOS image sequence 315 shows the IOS pattern by plotting absolute magnitude and ignoring the signal polarities.

With respect to FIG. 4, shown is an image describing comparative 105 and ERG analysis according to various embodiments of the present disclosure. For example, FIG. 4 depicts ERG waveforms recorded under conditions described below. IOS (portion (A) of FIG. 4) and ERG (portion (B) of FIG. 4) were recorded from the same groups of frogs. Each tracing represents an average of 4 responses evoked by light flashes of progressively brighter intensities over 5.0 log unit (log I/Iₘₐₓ) as indicated by the legend. Portion (C) of FIG. 4 depicts a normalized magnitude and portion (D) of FIG. 4 depicts a time-to-peak of an ERG a-wave, b-wave, and confocal-IOS plotted as a function of stimulus strength.

Experiments were designed to determine the physiological source of confocal-IOS by comparing IOS imaging and ERG recording. Graph 403 shows representative IOS magnitude dynamics elicited by 9 different stimulus strengths over a 5 log unit range. Graph 406 illustrates ERG waveforms recorded under the same conditions. The amplitude of the a-wave was measured from baseline to trough. The amplitude of the b-wave was measured from the a-wave trough to b-wave peak. IOS and ERG signals may not be measured simultaneously. Rather, they may be recorded under the same experimental conditions (same stimulus/illumination light) and in the same experimental specimen (the same eye). Both IOS and ERG signals were averaged based on 4 trials/eyes. For the first and third trial/eye, IOSs was first recorded, then ERG. For the second and fourth trial/eye, the order was changed to ERG recording first, followed by IOSs. In this way, differences in experiment conditions could be minimized between IOS and ERG recordings. It was typically observed that the IOS occurred almost immediately after the stimulus delivery, reaching peak magnitude within 150 ms. To compare time courses of IOS and ERG dynamics, ERG a-wave, b-wave, and IOS magnitudes were normalized as shown in graph 409. The amplitude of the b-wave first increased almost linearly with the gradual increased intensity of the stimulus, reached a maximum and then decreased as light intensity became higher than −1.5 log units. The a-wave is widely accepted as a measure of photoreceptor function 40. At low stimulus light intensities (below −3 log units), a-wave amplitude increased slowly with increased stimulus intensity, whereas it increased much faster when the light intensity was above −3 log units. Maximum a-wave amplitude was found at the light intensity of −0.5 log units, ten times higher than the maximum of −1.5 log units for the b-wave. As depicted in graph 409, the overall trend of IOS magnitude was quite consistent with that of a-wave amplitude, including the threshold and maximum response. This suggests that confocal-IOSs predominantly originate from retinal photoreceptors. Time-to-peak values of the IOS and ERG recordings also show similar dependency on stimulus intensity, decreasing as the light intensity increased, as shown in graph 412.

Moving on to FIG. 5, component 503a shows retinal structure before laser damage. Portion (A1) of FIG. 5 depicts a retinal structure of a normal frog eye and portion (B1) of FIG. 5 depicts the same retinal area after laser injury. Portion (A2) depicts a three dimension IOS image with a full field stimulus before (A2) and after (B2) laser injury. The corresponding overall IOS distribution images is obtained by smoothing, shown in portions (A3) and (B3) of FIG. 5. Scale bars 512a-f represent scale of 50 μm.

A full field stimulus with moderate intensity (at −1.5 log units) was applied to conduct confocal-IOS imaging. The corresponding three-dimensional (3D) surface envelope of the IOS image recorded within 0.1 s after stimulus delivery is illustrated in component 503b. For better visualization of the overall IOS distribution pattern, the IOS image may be smoothed using a mean filter (kernel size 15 μm×15 μm). A relatively homogeneous signal distribution pattern i shown with respect to component 509a.

In order to demonstrate the feasibility of detecting localized retinal damage, a 30-μm lesion was introduced after a control test depicted in components 503a and 503b. Thirty minutes after the laser exposure, the same full field stimulus was applied to this retinal area. From the structural images of component 503a and component 503b, visible changes are barely observable. However, IOS images with full field stimulus showed a signal-absent slit area located at the place where the laser damage was introduced, as depicted in component 506b. By using the smoothing method described above, the IOS magnitude image shown in component 509b showed a clear 30-μm-wide rectangle of markedly reduced signal. Therefore, our experiment indicated that rapid line-scan IOS imaging of intact frogs could be used for in vivo investigation of this localized retinal lesion.

Accordingly, a rapid line-scan confocal imager may be employed to achieve cellular resolution IOS imaging of retinal photoreceptors in vivo. The confocal-IOS patterns show tight correlation with localized retinal stimulation, as depicted in FIG. 3. A spatiotemporal filtering algorithm may be employed to separate stimulus-evoked fast IOS response from blood flow. Given that blood flow could induce significant optical fluctuation independent of retinal stimulation and blood flow, an associated artifact may be readily excluded by dynamic threshold rejection. This spatiotemporal filtering assumes that blood flow associated optical changes at any one location are consistent before and after stimulus delivery. Although it is possible that retinal stimulation may produce hemodynamic changes in the blood vessel area, such changes may not be detected in short (0.8 s) after-stimulus recording epoch.

Comparative ERG measurements were conducted to investigate physiological sources of the confocal-IOS. The experiments revealed tight correlation between the IOS response and ERG a-wave. Both magnitudes and time-courses of the IOS and a-wave showed similar responses to stimulus intensity changes. The time-to-peak of IOSs fell between the a-wave and b-wave. The a-wave leading edge is dominated by retinal photoreceptors and the later phase is truncated by electrophysiological response of inner retinal neurons, particularly ON bipolar cells. By recording a pure photoreceptor response, i.e., wherein post-photoreceptor neurons are blocked, the a-wave should take more time to return the baseline, which results in longer time to reach peak compared with the a-wave of standard ERGs41-43. From this perspective, if we assume the fast IOSs originate from retinal photoreceptors, the measured time-to-peak of the (OS should be longer than that of the standard a-wave, but shorter than b-wave, which is consistent with experimental results. Therefore, the confocal-IOSs may originate mainly from retinal photoreceptors. In addition, because of the frog eye's high numerical aperture (0.4), the axial resolution of confocal-IOS imaging was estimated at ˜10 μm. This resolution may be sufficient to distinguish the photoreceptors from other retinal layers. Previous studies with isolated photoreceptor outer segments and isolated retinas have demonstrated transient IOSs associated with phototransduction. Both binding and release of G-proteins to photo-excited rhodopsin might contribute to the positive (increased) and negative (decreased) IOSs. Localized biochemical processes might produce non-homogeneous light intensity changes, i.e., positive and negative signals mixed together.

A laser-injured frog model was used to validate confocal-IOS identification. By inducing localized retinal lesions through green laser exposure, it is demonstrated that confocal-IOS imaging can provide high transverse resolution, at least 30 μm. Based on early investigations of laser damage in other animal models, it is estimated that laser exposure could produce severe photoreceptor damage.

As may be appreciated, development of high resolution confocal-IOS imaging can lead to reliable physiological assessment of individual retinal photoreceptors. This prospect is particularly important for rods, known to be more vulnerable than cones in aging and early AMD, the most common cause of severe vision loss and legal blindness in adults over 50. Early detection and reliable assessment of medical interventions are key elements in preventing or slowing the progress of AMD associated vision loss. Both morphological and functional tests are important for reliable detection of AMD. Currently, there is no established strategy to allow objective assessment of retinal dysfunction at high resolution to allow direct comparison between localized physiological and morphological abnormalities in early AMD or other eye diseases. Confocal-IOS imaging will enable concurrent morphological and functional assessment of localized retinal dysfunctions in vivo. Further, it can be combined with technologies that assess structure and function of the photoreceptor support system that is affected even earlier in AMD. This combination could revolutionize the study, diagnosis and therapy assessment of AMD.

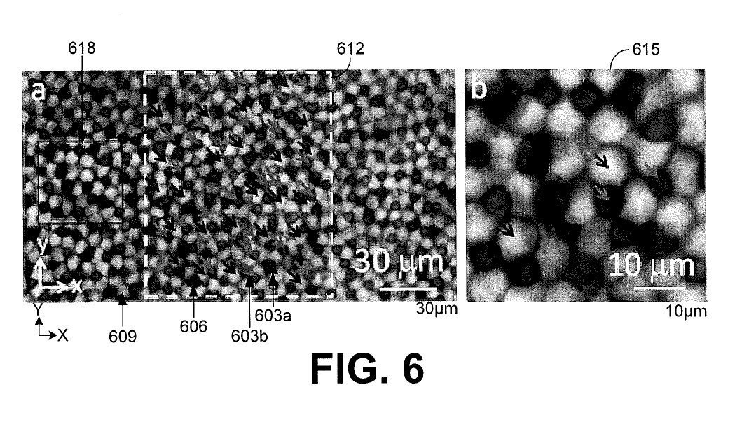

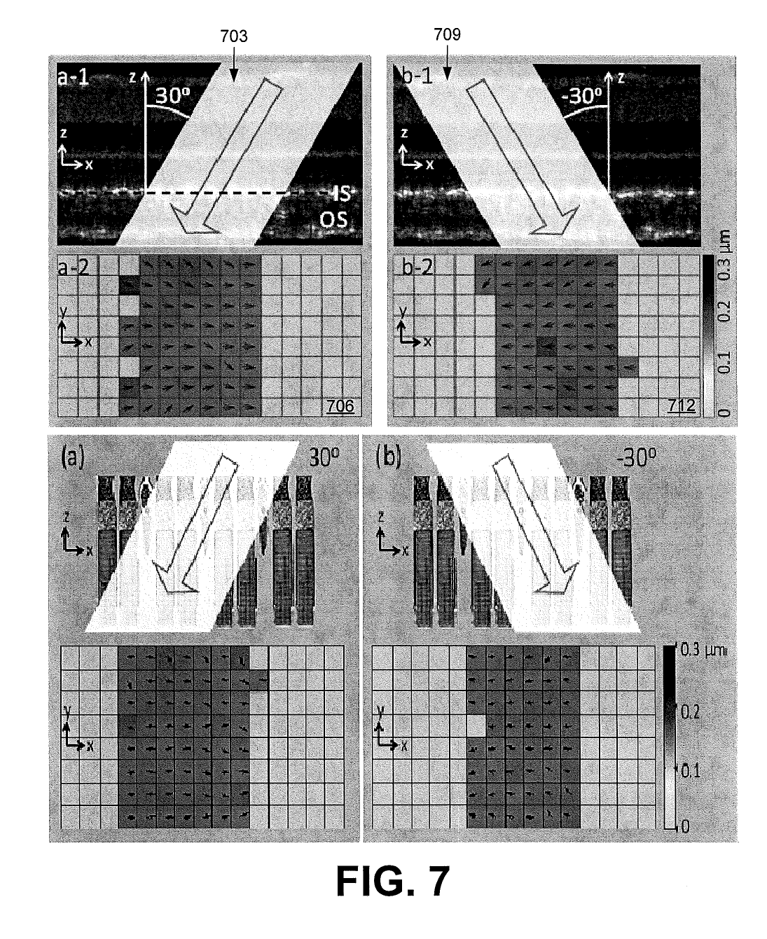

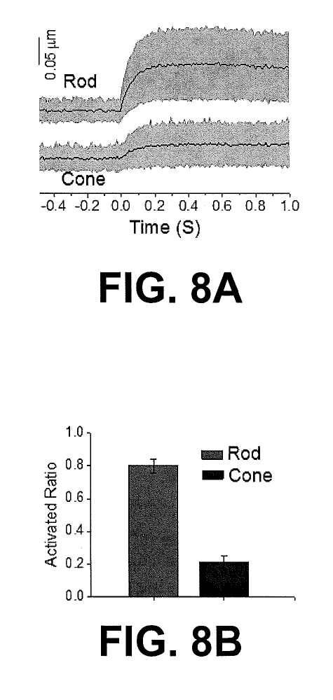

Turning next to FIG. 6, shown is a near infrared (NIR) image of frog photoreceptors according to various embodiments of the present disclosure. Shown in FIG. 6, arrows 603a and 603b point to rods 606 and cones 609, respectively. Box 612, depicted using a white dashed window, illustrates a stimulus pattern. Twenty-five rods 606 and cones 609 were randomly selected for obtaining the curve depicted in FIG. 8b. Further, FIG. 6 depicts an enlarged portion 615 of the area specified by the rectangle 618 in FIG. 6.

Stiles-Crawford effect (SCE) describes that luminous efficiency is dependent on incident light direction relative to eye axis. The retina is more sensitive to the light entering the center of the pupil, i.e., parallel light relative to eye axis, than that passing through the periphery, i.e., oblique light illumination. The SCE is exclusively observed in a cone system, which can benefit good vision quality by suppressing the intraocular stray light associated with wide pupil under a photopic situation and can act as a biomarker for quantitative assessment of functional integrity of cones 609. In contrast, the SCE is not detected in a rod system which dominates scotopic vision. Early SCE studies have been predominately based on psychophysics methods and, therefore, biophysical mechanisms underlying rod 606 and cone 609 discrimination is still unclear. Dynamic near infrared (NIR) light imaging may be employed to explore transient phototropic (e.g., directional) changes in individual rods 606 and cones 609. High-spatial (μm) and high-temporal (ms) resolution monitoring reveals that the majority (˜80%+) of rods 606 could rapidly move toward the direction of oblique stimulus light, while such directional movement was negligible in cones 609. This observation suggests that transient phototropic adaptation may quickly compensate for the loss of luminous efficiency in rods due to oblique stimulation. In contrast, it may take a long time, for example, at least tens of seconds, for cone adaptation to occur. The observed transient directional change of a retinal rod not only provides insight in better understanding of the nature of vision, but also promises an optical biomarker to allow non-invasive identification of rod dysfunction which is known to be more vulnerable than cones in aging and early age-related macular degeneration (AMD), the most common cause of severe vision loss and legal blindness in adults over 50.

FIG. 6 shows the NIR (800-1000 nm) image of an isolated frog retina acquired by a transmission microscope (see supplementary information for details). The NIR light was out of the sensitivity spectrum of the retina, and thus allowed stimulation artifact free observation of retinal photoreceptors. Frog retinas were selected in the non-limiting example of FIG. 6 because of several reasons. First, relatively large size of frog (compared to mouse or other mammalians) photoreceptors allows unambiguous observation of individual photoreceptors. Second, the diameter of frog rods (˜5-8 μm) is significantly different from that of the cones (˜1-3 μm) and rod and cone photoreceptors can be directly separated based on their cellular diameters. Third, rods 606 and cones 609 numbers are roughly equal in frog retinas, and thus unbiased analysis of rod and cone systems can be readily achieved. Fourth, the preparation procedure of freshly isolated living frog retinas has been established for functional study of retinal cells.

Moving on to FIG. 7, shown is an image depicting oblique stimulus-evoked photoreceptor displacements according to various embodiments of the present disclosure. In portion (a-1) of FIG. 7, a stimulus light was delivered at 30° relative to the photoreceptor axis. The retinal cross-section image was acquired by a high resolution OCT. In portion (a-2) of FIG. 7, shown as localized retinal displacements, corresponding to the 30° stimulation. The color of each sub-image (square window) indicates the displacement magnitude, while arrows indicate the displacement direction. Portion (b-1) of FIG. 7 relates to the stimulus light that was delivered at 30° relative to the photoreceptor axis. Portion (b-2) of FIG. 7 depicts localized retinal displacements, corresponding to the 30° stimulation.

In order to test transient directional response of retinal photoreceptors, a white (450-650 nm) light flash (5 ms) was used to stimulate the retina, with a rectangular box 612 and oblique illumination angle at 30° relative to the normal axis of retinal surface, as depicted in region 703 in FIG. 7. Dynamic localized registration between the post-stimulus images and pre-stimulus baseline disclosed localized movements in the retina activated by the oblique stimulus light (see Supplementary Information for details). As shown in region 706 of FIG. 7, which was recorded at 200 ms after the stimulus delivery, the stimulus activated retina shifted to right, i.e., towards the direction of the oblique stimulation. In order to demonstrate the reliability of the phototropic response, the incident angle of the flash stimulus light was switched to −30° (region 709), 5 minutes after the recording illustrated in region 706. Region 712 shows the movement map recorded at 200 ms after the −30° stimulus delivery, from the same retina used in region 706. It was observed that the stimulated retina shifted toward left (region 712), i.e., in the opposite direction compared to region 706. Comparative recording of the 30° and −30° stimuli verified transient movements, which were unambiguously dependent on the incident direction of the stimulus light, of retinal photoreceptors in the retina.

In order to quantify transient phototropic changes in rod and cone systems, displacements of individual rods and cones may be estimated. For example, twenty-fix cones 609 (FIG. 6) and twenty-five rods 606 (FIG. 6) were selected randomly from each trial within the stimulus area. The level-set method was used to identify morphological edge of each cone 609 or rod 606, and weight centroids of individual cones 609 and rods 606 were calculated.

Moving on to FIG. 8A, shown are average displacements of twenty-five rods 606 (FIG. 6) and twenty-five cones 609 (FIG. 6) located within the stimulus windows. FIG. 8A depicts average displacement curve of twenty-five rods 606 (FIG. 6) and cones 609 (FIG. 6). The gray shadow indicates the standard deviation. In FIG. 8B, shown is a comparison of activated ratio between rods and cones. In the non-limiting example of FIG. 8B, six trials were used. For each trial, twenty-five rods 606 and cones 609 were randomly selected.

As shown in FIG. 8A, the displacement of rods 606 occurred almost immediately (<10 ms) and reached magnitude peak at ˜200 ms. The magnitude of rod displacement (0.08 μm) was significantly larger than that (0.024 μm) of cone displacement. From the prestimulus period, we used three standard deviations plus the mean to define a threshold for distinguishing rods 606 and cones 609. If the displacement magnitude was equal to or greater than the threshold, then the rod 606 or cone 609 was defined as active. Otherwise, the rod 606 or cone 609 was defined as silent. Among those selected rods 606, 76% were actively associated with the oblique stimulation, while only 20% among those cones 609 were active. To further confirm the phototropic displacement was dominant in rods 606, measurement using 6 retinas was repeated, under identical experimental conditions. For example, twenty-five rods 606 (and/or cones 609) were randomly selected from each stimulated retina to evaluate active ratios of rods and, as shown in FIG. 8B, the active ratio of rods was 80%±4%, while 20%±4% of cones were activated, indicating that displacement was dominant in rods 606.

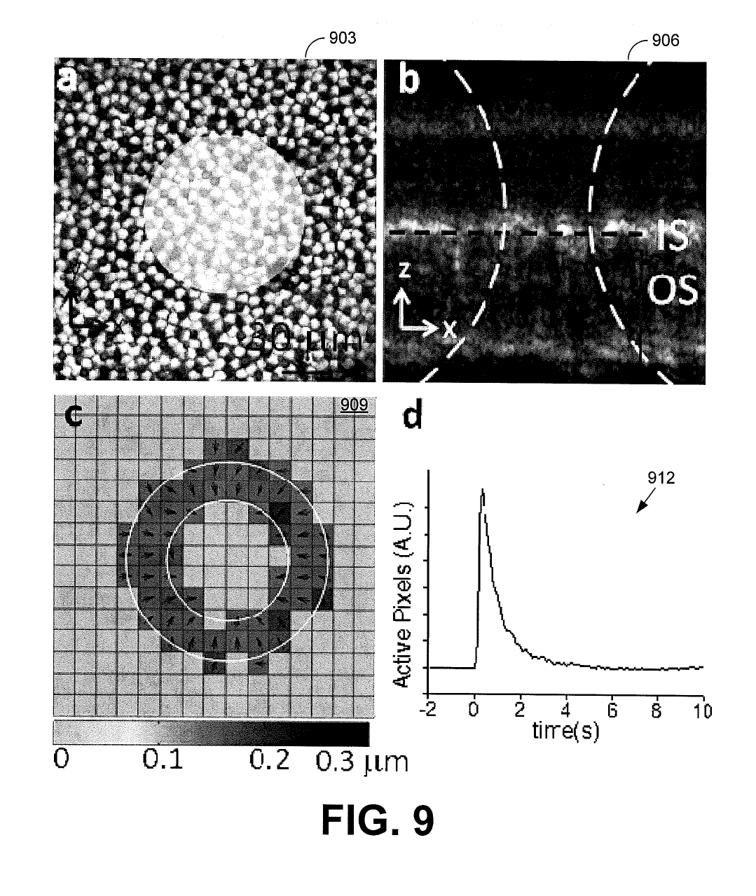

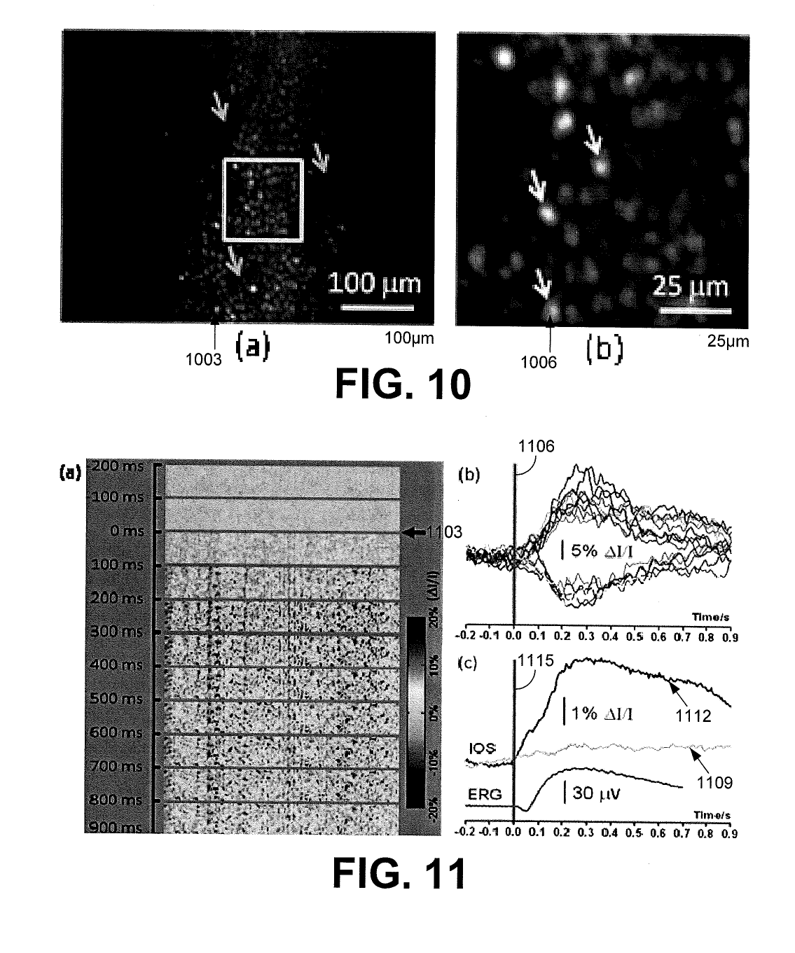

Moving on to FIG. 9, shown are the results of an investigation of transient photoreceptor displacement correlated with circular stimulation according to various embodiments of the present disclosure. Portion (a) of FIG. 9 depicts an NIR image of retinal photoreceptors and stimulus pattern. Portion (b) of FIG. 9 depicts a Gaussian shape pattern of the stimulus light in cross-section view of the retina. The circular stimulus was converged to the IS and then became divergent at the OS. Portion (c) of FIG. 9 depicts the map of the photoreceptor displacements recorded after the stimulus delivery. Portion (d) of FIG. 9 depicts the number of active pixels as a function of time. As a non-limiting example, the recording time was 12 seconds.

In addition to the aforementioned oblique stimulation, transient photoreceptor displacements may be tested in the retina activated by a circular stimulus pattern 903, with a Gaussian profile in the axial plane, as depicted in region 906. The circular aperture was conjugated to the focal plane of the imaging system. When the mosaic pattern of photoreceptors was clearly observed, the focal plane was around the photoreceptor inner segment (IS). Therefore, at the more proximal position, i.e., of the outer segment (OS), the stimulus light become diverged, as shown in region 906. Under this condition, only photoreceptors at the periphery of the stimulus pattern showed transient displacements towards the center of the circular spot, as depicted in region 909. The active pixel numbers were plotted as a function of the time in graph 912. Rapid displacement occurred almost immediately (<10 ms) after the stimulus delivery, and reached the magnitude peak at ˜200 ms, shown in region 906. The recovery phase of the phototropic change lasted ˜2 seconds, shown in graph 912. It was consistently observed that the stimulus-evoked displacement was rod dominant. The method described above with respect to FIGS. 8A-B for distinguishing rods and cones may be employed, wherein six trials may be conducted under the same conditions. Within the annular area specified in region 909, twenty-fix rods 606 and cones 609 may be randomly selected. 74%±6% of rods were activated, while 24%±5% of cones were activated. The off-center and on-surround displacement pattern (region 909) evoked by the circular stimulation was consistent to the observation in the retina activated by oblique stimuli (FIG. 7). At the center area, the stimulus light impinged the photoreceptor without directional dependence. At the edge of the stimulus, the Gaussian-shape light distribution (region 906) evoked directional displacements.

Accordingly, high spatial temporal resolution imaging reveals transient phototropic response in the retina stimulated by oblique stimuli (FIG. 6 and FIG. 7). The transient phototropic response was dominated by retinal rods 606. Although a small portion (˜20%) of cones 609 also showed transient response (FIG. 8B) correlated with the retinal stimulation, possible artifacts may not be excluded due to adjacent rod movements. Experimental results suggest that transient phototropic adaptation may quickly compensate for the loss of luminous efficiency in rods due to oblique stimulation. In contrast, it may take long time, for example, at least tens of seconds, for cone adaptation to occur. In other words, rapid (onset: ˜10 ms; time-to-peak: ˜200 ms) phototropic adaptation in retinal rods 606 is too quick for the SCE to be detected by conventional psychophysics methods based on brain perception. The distinct SCE of rods and cones is consistent to the function of rod and cone systems. Cones 609 and rods 606 are predominantly responsible for scotopic (night) and photopic (daylight) conditions. For the photopic vision when the light entering the eye is ample, the evolution may have the SCE developed in retinal cones to enhance image quality by rejecting stay light and improving spatial resolution. For the scotopic vision with dim light, the imperative is to collect enough light to ensure retinal sensitivity. Therefore, the rapid phototropic displacement of retinal rods can be valuable to ensure light efficiency for scotopic vision.

Circular pattern stimulation further confirms the transient rod displacement (FIG. 9). In addition, the observed off-center and on-surround pattern (FIG. 9) may imply early involvement of the photoreceptors in contrast enhancement and center-surround antagonism in the retina. In general, it is believed that the center-surround antagonism is initiated by horizontal cells and/or Amacrine cells. In contrast, experimental results suggest that the discrepancy of the incident angle between the surround and the center of the Gaussian illumination (region 906) can evoke directional displacement only at the surround. Such edge-enhanced pattern of photoreceptor activity may suggest early involvement of the photoreceptors in center-surround antagonism directly. Further investigation is necessary to understand how the rod distinguishes the incident angle at the molecule level. The observed transient directional change of retinal rod not only provides insight in better understanding of the nature of vision, but also promises an optical biomarker to allow non-invasive identification of rod dysfunction at early staged of AMD. Structural biomarkers, such as drusen and pigmentary abnormalities in the macula, have provided valuable information for AMD test. However, the morphological only fundus examination may not be enough. Combined structural and functional tests re desirable for early detection of AMD. Psychophysical methods, such as Amsler grid test, visual acuity, and hyperacuity perimeter, are practical in clinical applications, but they involve extensively higher order cortical processing. Therefore, they do not provide exclusive information on retinal function and lacks the sensitivity in detecting early AMD. Reliable objective assessment of retinal function, particularly the rod system that is known to be more vulnerable than cone at the onset stage of AMD. Further understanding of the rod dominant phototropic effect may provide a high resolution methodology to achieve accurate identification of rod dysfunction, and thus to allow early detection and easy treatment evaluation of AMD.

Moving on to FIG. 10, is a drawing of an in vivo image (500×400 pixels) of a frog retina according to various embodiments of the present disclosure. Functional evaluation is important for retinal disease detection and treatment evaluation. It is well known that many eye diseases can cause pathological changes of photoreceptors and/or inner retinal neurons that ultimately lead to vision losses and even complete blindness. Different eye diseases, such as age-related macular degeneration (AMD), retinitis pigmentosa (RP), glaucoma, etc., are known to target different types of retinal neurons, causing localized lesions or cell losses. Electroretinogram (ERG), focal ERG, multifocal ERG, perimetry, etc., have been established for functional examination of the retina. However, spatial resolution and signal selectivity of the ERG and perimetry may not be high enough to provide precise identification of localized retinal dysfunctions. While it is possible to combine morphological (such as high resolution OCT) with functional (such as ERG) evaluation to improve retinal disease study and diagnosis, conducting these separate measurements is time-consuming and cost-inefficient. Moreover, morphological and functional changes of the retina are not always correlated. Given the delicate structure and complicated functional interaction of the retina, detection of localized dysfunction requires a method that can examine stimulus-evoked retinal functional activities at high spatial and temporal resolutions.

Intrinsic optical signal (IOS) imaging may provide a non-invasive method for concurrent morphological and functional evaluation of the retina. Several imaging techniques, such as fundus cameras, adaptive optics ophthalmoscopes, and/or optical coherence tomography (OCT) imagers have been explored to detect transient IOSs associated with retinal stimulation. In principle, both stimulus-evoked retinal neural activity and corresponding hemodynamic and metabolic changes may produce transient IOSs associated with retinal stimulation. While hemodynamic and metabolic changes associated slow IOSs can provide important information in functional assessment of the visual system, they are relatively slow and cannot directly track fast neural activities in the retina. Fast IOSs, which have time courses comparable to electrophysiological kinetics, are desirable for direct evaluation of the physiological health of photoreceptors and inner neurons. Using freshly isolated frog retinas, a series of experiments may be conducted to validate high-spatial (sub-cellular) and high-temporal (ms) resolution imaging of stimulus-evoked fast IOSs in the retina. As discussed below, the feasibility of in vivo imaging of fast IOSs in the retina of intact frogs is shown.

During IOS recording, the frog eye was continuously illuminated by the NIR light. With the line-scan confocal system, high resolution in vivo images revealed individual blood vessels 1003 (also shown in the arrowheads of portion (a) of FIG. 10) and photoreceptors 1006 (also shown in the arrowheads of portion (b) of FIG. 10).

Moving on to FIG. 11, portion (a) of FIG. 11 depicts a representative spatial IOS image sequence. Each illustrated frame is an average over 100 ms interval (20 frames). The black arrowhead 1103 indicates the onset of the 10 ms green flash stimulus. 200 ms pre-stimulus baseline and 900 ms post-stimulus IOS recordings are shown. Portion (b) of FIG. 11 is representative of IOS responses of individual pixels randomly selected from the image area. The bar 1106 indicates the stimulus onset and duration. Portion (c) of FIG. 11 depicts a top black trace showing IOS magnitude (i.e., absolute value of the IOS) averaged over the whole image area, corresponding to the image sequence shown in portion (a) of FIG. 11. The light trace 1109 shows one control experiment without stimulation. The dark trace 1112 below shows concurrent frog ERG. The bar 1115 indicates the stimulus delivery.

To ensure high temporal resolution for IOS recording, one sub-image (250×50 pixels) area was selected to achieve high-speed (200 frames/s) measurement. During the IOS imaging, retinal ERG response was recorded simultaneously. FIG. 11 represents retinal IOS responses recorded in intact frogs. In portion (a) of FIG. 11, high spatial resolution images revealed both positive (increasing) and negative (decreasing) IOS responses, with sub-cellular complexities. Portion (b) of FIG. 11 shows (OS dynamics of individual pixels selected randomly from the image area. It is observed that the peak IOS magnitude of sub-cellular locations was up to 20% ΔI/I, where ΔI was the light intensity change and I was the background light intensity. As shown in portion (b) of FIG. 11, the positive and negative IOSs had comparable time courses, in terms of time-delay and time-to-peak (relative to stimulus onset). By ignoring the signal polarity, portion (C) of FIG. 11 shows averaged IOS magnitude (i.e., absolute value of the IOS), and corresponding retinal ERG. As shown in portion (c), fast IOSs occurred almost immediately (<10 ms) and reached the peak magnitude within ˜300 ms after the stimulus onset. Comparable retinal ERG was observed.

Accordingly, the feasibility of in vivo imaging of retinal activation is demonstrated with intact frogs. A rapid line-scan confocal ophthalmoscope may be constructed to achieve high spatiotemporal resolution imaging of fast IOSs. By rejecting out-of-focus background light, the system resolution was significantly improved in comparison with our previous flood-illumination imager. High resolution confocal images revealed individual frog photoreceptors in vivo. Robust IOSs were clearly imaged from the stimulus activated retina, with sub-cellular resolution. High resolution images revealed fast IOSs that had time courses comparable to retinal ERG kinetics. The experiment indicates that rapid line-scan IOS imaging of intact frogs provides a simple platform for in vivo investigation of fast IOSs correlated with retinal activation. It is anticipated that future study of the fast IOSs can provide insight for developing advanced instruments to achieve concurrent morphological and functional evaluation of human retinas, with high spatial resolution to differentiate individual retinal cells.

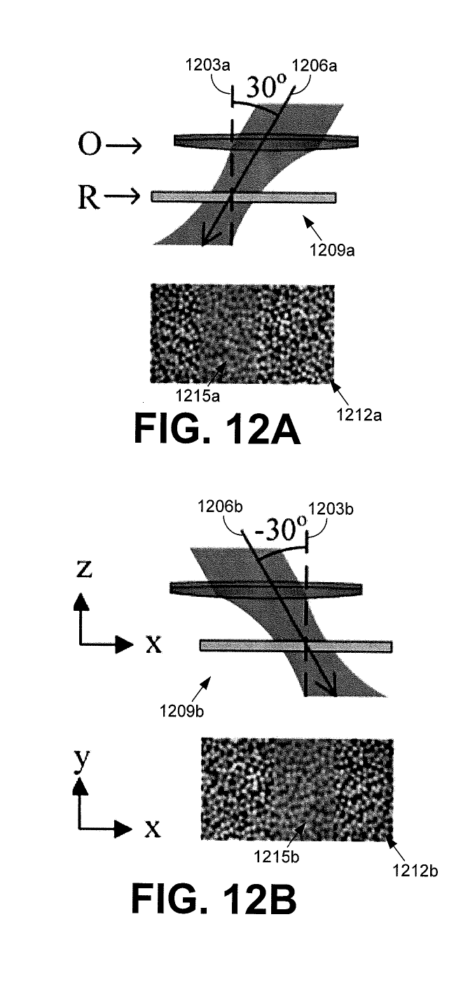

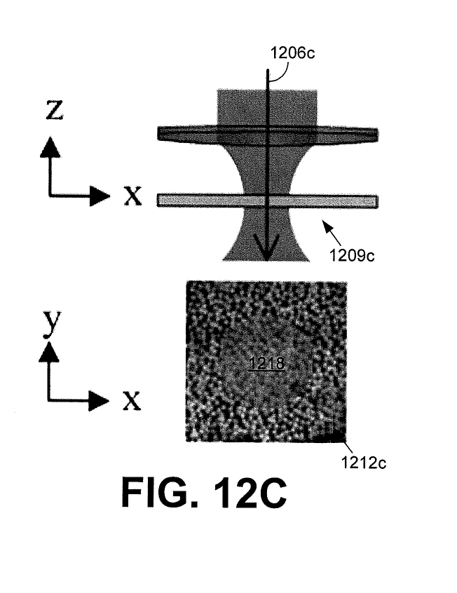

Moving on to FIGS. 12A-C, shown are schematic diagrams of stimulation patterns, wherein “O” denotes “objective” and “R” denotes “retinal.” Black dash lines 1203a and 1203b indicate the normal axis of retinal surface. Red solid lines 1206a, 1206b, and 1206b indicate the incident directions. Top panels 1209a, 1209b, and 1209c are cross-section views (transverse or x-z plane) and bottom panels 1212a, 1212b, and 1212c are en face views (axial or x-y plane). FIG. 12A depicts a rectangular stimulus 1215a with a 30° incident angle with respect to the normal axis of the retinal surface. FIG. 12B depicts a rectangular stimulus 1215b with a −30° incident angle. FIG. 12C depicts a circular stimulus 1218 with 0° incident angle. The retina was placed with the ganglion cell layer facing toward the objective.

In the non-limiting example of FIGS. 12A-C, both frog (Rana pipiens) and mouse (Mus musculus) retinas were used to demonstrate the transient phototropic adaptation in the retina. Frog retinas may be selected as primary specimens for several reasons. First, the relatively large size of frog (compared to mouse or other mammalian) photoreceptors allows unambiguous observation of individual photoreceptors. Second, the diameter of frog rods (˜5 to 8 μm) is much larger than cones (˜1 to 3 μm) and thus, rod 606 (FIG. 6) and cone 609 (FIG. 6) photoreceptors can be easily separated based on their cellular diameters. Third, rod 606 and cone 609 numbers are roughly equal in frog retinas and thus, unbiased analysis of rod and cone cells can be readily achieved. Briefly, the frog was euthanized by rapid decapitation and double pithing. After enucleating the intact eye, the globe was hemisected below the equator with fine scissors. The lens and anterior structures were removed before the retina was separated from the retinal pigment epithelium.

Mouse retinas were used to verify the transient phototropic adaptation in mammalians. Five-month-old wild-type mice, which have been maintained for more than twenty generations from an original cross of C57Bl/6J to 129/SvEv, were used. The rd1 allele that segregated in the 129/SvJ stock was removed by genetic crossing and verified. Briefly, after the eyeball was enucleated from anesthetized mice, the retina was isolated from the eyeball in Ames media and then transferred to a recording chamber. During the experiment, the sample was continuously superfused with oxygenated bicarbonate-buffered Ames medium, maintained at pH 7.4 and 33° C. to 37° C.

To generate the data of FIGS. 12A-C, the imaging systems of FIG. 1A or FIG. 1B, or variations thereof, may be employed. For example, an imaging system based on a NIR digital microscope that has been previously used for functional imaging of living retinal tissues may be employed. A fast digital camera, such as a Neo sCMOS (Andor Technology) with a pixel size 6×6 μm² may be employed for retinal imaging. A 20× water immersion objective with 0.5 NA was used for frog experiments. Therefore, the lateral resolution of the system was about 1 μm (0.61λ/NA). For mouse experiments, a 40× water immersion objective may be used with a 0.75 NA which has the lateral resolution of 0.7 μm. The imaging system may comprise, for example, two light sources: a NIR (800 to 1000 nm) light for retinal imaging and a visible (450 to 650 nm) light-emitting diode (LED) for retinal stimulation. The duration of the visible flash may be set to 5 ms.

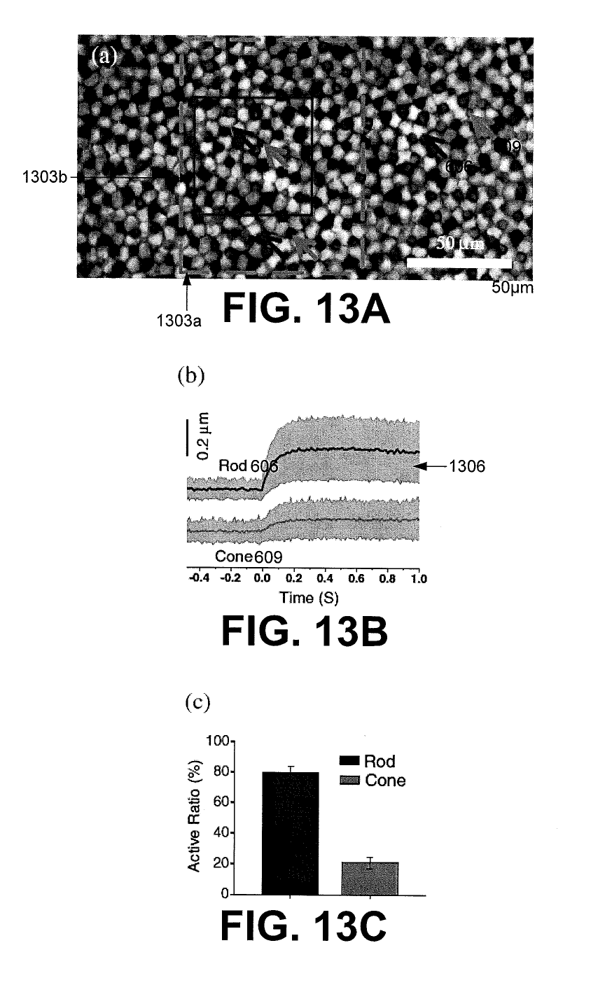

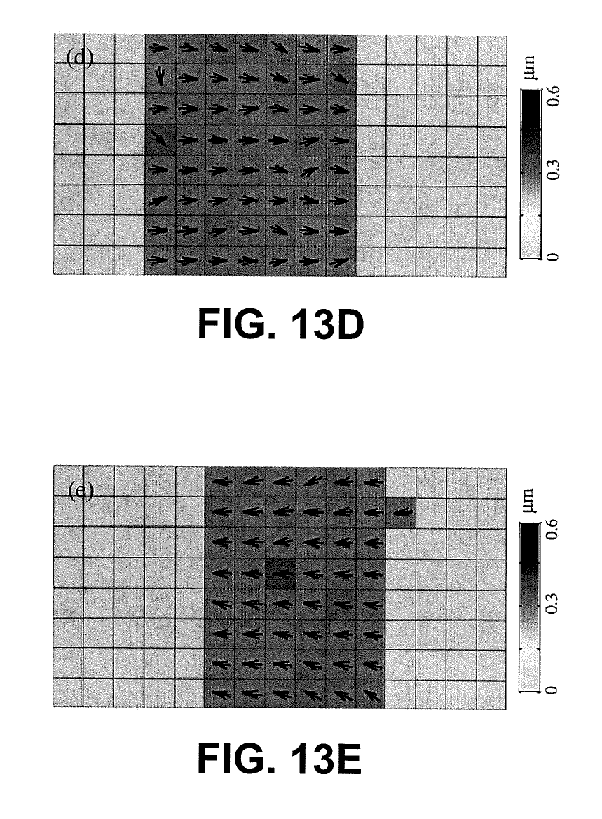

FIGS. 12A-C illustrate rectangular stimulus patterns with oblique incident angles (FIGS. 12A-B) and a circular stimulus pattern with perpendicular incident angle (FIG. 12C). FIGS. 12A-B were used for the experiments in FIGS. 13 and 15, and FIG. 14, respectively. All images of retinas in FIGS. 12-15 were acquired at 200 frames/s.

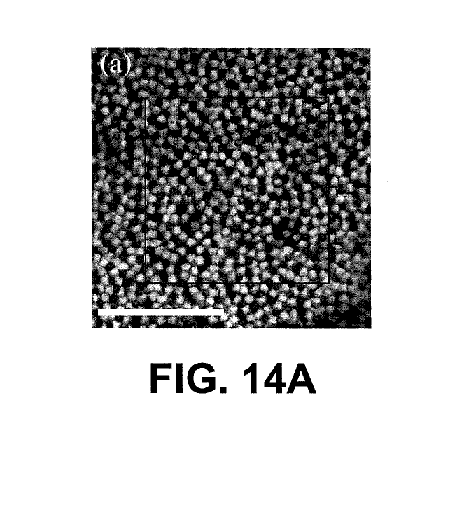

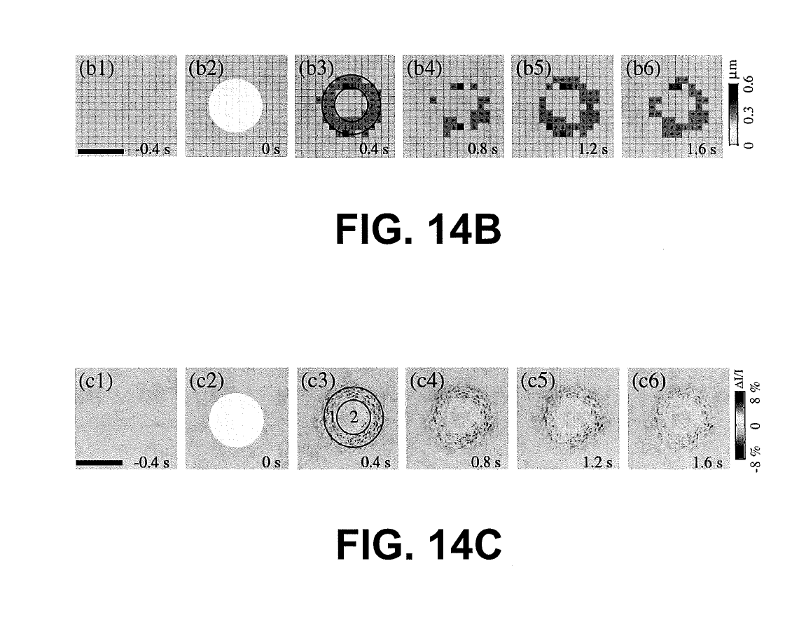

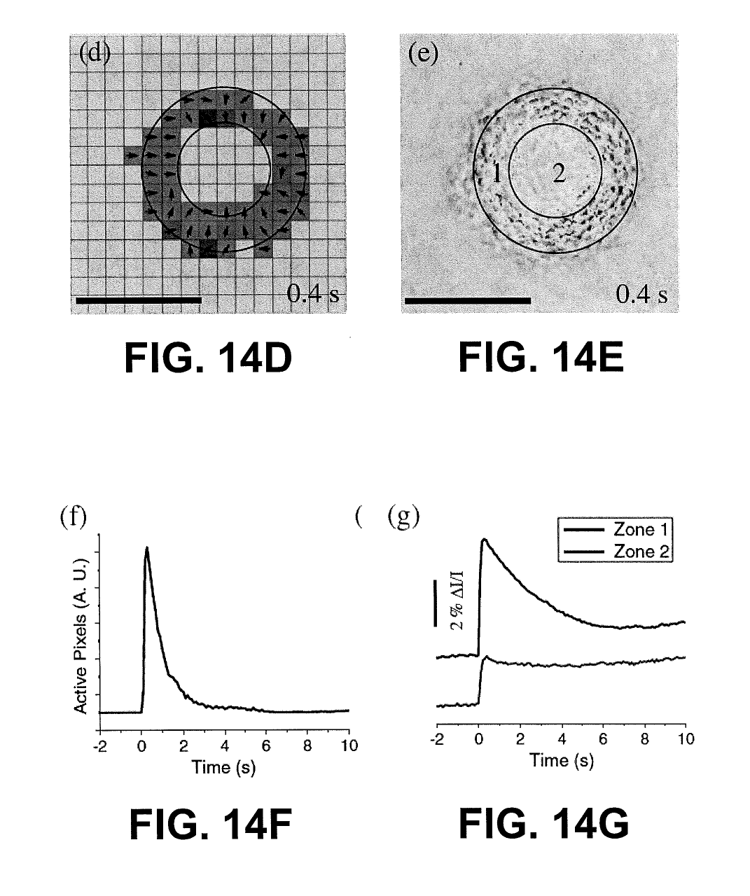

Moving on to FIGS. 13A-E shown are images depicting oblique stimulus-evoked photoreceptor displacements. FIG. 13A depicts a near infrared (NIR) image of a frog photoreceptor mosaic pattern. A first dashed window 1303a illustrates a stimulus area. A second dashed window 1303b indicates the area which displays a pair of pre- and post-stimulus images alternating repeatedly twenty times. Arrows point to rods 606 and 609 cones, respectively. FIG. 13B depicts the average displacement of twenty-five rods 606 and cones 609 which were randomly selected from the stimulus area. The shadow 1306 indicates the standard deviation. FIG. 13C depicts the active ratios of rods 606 and cones 609 at time 200 ms after the onset of the stimulus. In the non-limiting example of FIG. 13C, six trials were used. For each trial, twenty-five rods 606 and cones 609 were randomly selected. Thus, in each trial, the active ratio was calculated as the number of active rods or cones divided by twenty-five. In FIG. 13D retinal displacements associated with the 30° stimulus (See FIG. 14A) at 200 ms. In FIG. 13E, retinal displacements associated with the −30° stimulus at 200 ms. Each square in FIGS. 13D and 13E represents a 15×15 μm2 area of the retina. Transient displacements within the small square were averaged.

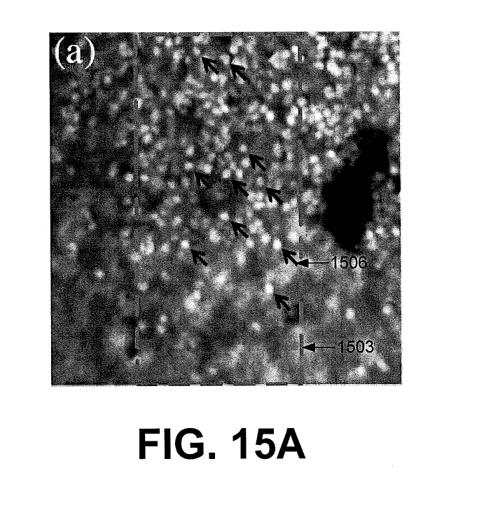

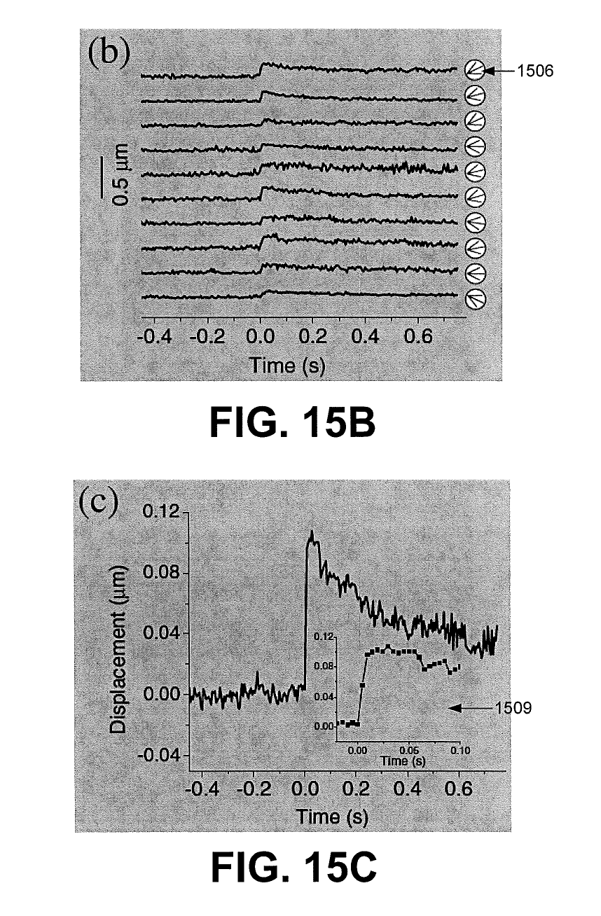

In order to quantify transient phototropic changes in rod and cone systems, the displacement of individual rods (FIG. 11B and FIG. 15B) and cones (FIG. 13B) may be calculated. The level-set method may be utilized to identify the morphological edge of individual rods and cones. Then, the weight centroid may be calculated dynamically, allowing accurate registration of the location of individual photoreceptors at nanometer resolution. The same strategy has been used in stochastic optical reconstruction microscopy and photoactivated localization microscopy to achieve nanometer resolution to localize individual molecules with photoswitchable fluorescence probes. The three-sigma rule was used to set up a threshold to distinguish silent and active photoreceptors. If the stimulus-evoked photoreceptor shifted above this threshold, then this photoreceptor was defined as active. Otherwise, it was defined as silent. Thus, the active ratio of the rods and cones could be obtained (FIG. 13C).

The activated photoreceptors may be displaced due to light stimulations [FIGS. 13C-D and FIG. 14B]. In order to quantify the photoreceptor displacements, the normalized cross correlation (NCC) between the poststimulus and prestimulus images may be calculated to estimate localized retinal movements. It is assumed that Ī(x, y) was the image acquired at the time point of tᵢ, where i=1, 2, 3, . . . was the image index and (x, y) was the pixel position. The first image Īₜ₁(x, y) may be denoted as the reference image. For the pixel at the position of (x₀, y₀) from the image Īₜ₁, there would be a horizontal shift Hₜ₁(x₀, y₀) (parallel to the x axis) and a vertical shift Vₜ₁(x₀, y₀) (parallel to the y axis) compared to the reference image. At the position of (x₀, y₀) from the image Īₜ₁, a subwindow Wₜ₁ (m×m pixels) may be denoted by:

$${W}_{t 1} ( {x}_{0} , {y}_{0} , u , v ) = {\overline{I}}_{t 1} ( {x}_{0} - \frac{m - 1}{2} + u , {y}_{0} - \frac{m - 1}{2} + v ) . \qquad ( eq . 4 )$$

where u=1, 2, 3, . . . m, and v=1, 2, 3, . . . m. m is set wherein m=13 (corresponding to 3.9 μm at the retina). This window is at the level of individual cells (cone: 5 to 8 μm and rod: 1 to 3 μm). A corresponding subwindow of the reference image at the position of (x₁, y₁) is selected and the subwindow is denoted by:

$${W}_{t 1} ( {x}_{1} , {y}_{1} , u , v ) = {\overline{I}}_{t 1} ( {x}_{1} - \frac{m - 1}{2} + u , {y}_{1} - \frac{m - 1}{2} + v ) . \qquad ( eq . 5 )$$

The correlation coefficient may be calculated between two image matrices defined by eqs. 4 and 5 via:

$${CC}_{t 1} ( {x}_{0} , {y}_{0} , {x}_{1} , {y}_{1} ) = \frac{{\sum }_{u = 1}^{m} {\sum }_{v = 1}^{m} [ {W}_{ti} ( {x}_{0} , {y}_{0} , u , v ) - \overline{{W}_{ti}} ] [ {W}_{t 1} ( {x}_{1} , {y}_{1} , u , v ) - \overline{{W}_{t 1}} ]}{\begin{matrix}{\{ {\sum }_{u = 1}^{m} {\sum }_{v = 1}^{m} [ {W}_{ti} ( {x}_{0} , {y}_{0} , u , v ) - \overline{{W}_{ti}} ] \}}^{0.5} \\ {\{ {\sum }_{u = 1}^{m} {\sum }_{v = 1}^{m} [ {W}_{t 1} ( {x}_{1} , {y}_{1} , u , v ) - \overline{{W}_{t 1}} ] \}}^{0.5}\end{matrix}} . \qquad ( eq . 6 )$$

where W̅ₜ̅ᵢ̅ is the mean of the matrix Wₜᵢ(x₀, y₀, u, v), and W̅ₜ̅₁̅ was the mean of the matrix Wₜ₁(x₀, y₀, u, v). We searched x₁ from x₀−k to x₀+k, and y₁ from y₀−k to y₀−k, where k was the searching size, set to be 3 (corresponding to 0.9 μm at the retina) here. Thus, we could find the position (x_{1 max}, y_{1 max}), where the value of correlation coefficient defined by eq. 6 is maximum. Therefore, the horizontal shift (parallel to x axis) and vertical shift (parallel to y axis) at the position (x₀, y₀) were obtained as:

Hₜᵢ(x₀,y₀)=(x₀−x_{1 max}) (eq. 7), and

Vₜᵢ(x₀,y₀)=(y₀−y_{1 max}) (eq. 8).

This may be rewritten as a complex number via:

Hₜᵢ+jVₜᵢ=Aₜᵢexp(jΦₜᵢ) (eq. 9),

where j is the imaginary unit, Aₜᵢ is the shift amplitude map (e.g., FIGS. 13D, 13E, and 14B] and Φₜᵢ is the direction map (e.g., the directions of arrows in FIGS. 13D, 13E, and 14B). If

Aₜᵢ≠0 (eq. 10),

then the pixel (x₀, y₀) was displaced, thus defined as active. Therefore, the active pixel numbers could be plotted as a function of the time, as shown in FIG. 14F.

In order to test the effect of the phototropic adaptation on the IOS pattern associated with circular stimulus, representative IOS images are illustrated in FIG. 14C, with a unit of ΔI/I, where I is the background light intensity and ΔI reflects the light intensity change corresponding to retinal stimulation.

FIGS. 13A-E shows results of phototropic adaptation correlated with oblique light stimulation. FIG. 13A shows the photoreceptor mosaic pattern. Individual rods 606 and cones 609 could be observed. A rectangular stimulus with a 30° incident angle (FIG. 12A) is delivered to the retina. Within the stimulation area, photoreceptor displacements were directly observed in NIR images. In order to quantify transient phototropic changes in rod and cone systems, displacements of individual rods 606 and cones 609 may be calculated. FIG. 13B shows average displacements of twenty-five rods 606 and cones 609 randomly selected from the stimulus window.

The displacement of rods occurred almost immediately (<10 ms) and reached a magnitude peak at ˜200 ms. The magnitude of rod displacement (average: 0.2 μm, with maximum up to 0.6 μm) was significantly larger than that of cone displacement (average: 0.048 μm, with maximum of 0.15 μm). In addition, as shown in FIG. 13C, the active ratio of rods was 80%±4%, while 20%±4% of cones were activated. The observation indicated that the transient phototropic displacement was dominantly observed in rods.

In order to verify directional dependency of the phototropic adaptation, we used template matching with the NCC to compute non-uniform motion in the retina. As shown in FIG. 13D, the stimulus-activated retina shifted to right, i.e., toward the direction of the 30-deg oblique stimulation. In order to confirm the reliability of the phototropic response, the incident angle of the stimulus was switched to −30° (FIG. 12B), 5 min after the recording illustrated in FIG. 13D. FIG. 13E illustrates the transient movement corresponding to −30° stimulus at the same retinal area shown in FIG. 13D. It is observed that the stimulated retina shifted toward the left (FIG. 13E), i.e., in the opposite direction compared to FIG. 13D. Comparative recording of the 30° and −30° stimuli verified that transient photoreceptor movement was tightly dependent on the incident direction of the stimulus light.

In addition to the aforementioned oblique stimulation, FIG. 14A shows transient photoreceptor displacements activated by a perpendicular circular stimulus with a Gaussian profile in the axial plane FIG. 12B. The circular aperture was conjugate to the focal plane of the imaging system. Cones taper toward the outer segment (OS) and are shorter than rods, which imply that the OS pattern should have relatively larger extracellular space between photoreceptors when compared to the inner segment (IS) pattern. Therefore, when the tight mosaic pattern of photoreceptors (FIG. 13A) was clearly observed, the focal plane was around the photoreceptor IS. Hence, at the more distal position, i.e., the OS, the stimulus light was divergent and became oblique at the edge. However, at the central area, the stimulus light impinged the photoreceptor without directional dependence. Under this condition, only photoreceptors at the periphery of the stimulus pattern underwent displacement. FIGS. 14B and 14D not only confirmed this phenomenon but also revealed that peripheral photoreceptors shifted toward the center. The number of active pixels (eq. (10)) was plotted over time in FIG. 14F. The rapid displacement occurred almost immediately (<10 ms) after the stimulus delivery, reached the magnitude peak at ˜200 ms, and recovered at ˜2 s. It was consistently observed that the stimulus-evoked displacement was rod dominant. Utilizing the same methods employed in FIG. 13C, rod and cone displacements were quantitatively calculated. Within the annular area in FIG. 14D, twenty-five rods and cones were randomly selected for quantitative comparison. 74%±6% of rods were activated, whereas 24%±5% of cones were activated at 200 ms after the onset of stimulus (six samples).

It was speculated that the transient phototropic changes may partially contribute to stimulus-evoked IOSs, which promised a non-invasive method for spatiotemporal mapping of retinal function. IOS images shown in FIG. 14C confirmed the effect of IOS enhancement at the edge of the circular stimulus. The edge enhanced IOS response gradually degraded over time, which was consistent with the change of the photoreceptor displacement (FIG. 14B). In addition, both positive and negative IOS signals, with high magnitude, were observed at the periphery of the stimulus pattern. In contrast, the IOS signal at the stimulus center (Zone 1, FIG. 14E) was positive dominant, and the IOS magnitude was weaker than that observed at peripheral area (Zone 2, FIG. 14E). Moreover, time courses of IOS responses were different between Zone 1 and Zone 2, as depicted in (FIG. 14G). The central IOS curve (curve in FIG. 14G) more resembled the curve of the active pixel number (FIG. 14F), which suggested that transient phototropic change primarily contributes to the periphery IOSs.

With respect to FIGS. 15A-C, shown are stimulus-evoked photoreceptor displacements at the mouse retina. FIG. 15A depicts an NIR image of mouse photoreceptor mosaic. A 40× objective with 0.75 NA was used. The image size corresponds to a 60×60 μm2 area at the retina. The dashed rectangle 1503 indicates the oblique stimulation area. FIG. 15B depicts Displacements of ten photoreceptors over time. The stimulus was delivered at time 0. These ten photoreceptors are specified by arrows in FIG. 15A. Arrows in circles 1506 indicate the direction of the displacement at time 30 ms after stimulation. In FIG. 15C, the averaged displacement of ten photoreceptors is shown. The inset panel 1509 shows the same data within the time period from −0.02 to 0.1 s.

In order to verify the transient phototropic changes in mammalians, we have conducted a preliminary study of mouse retinas with oblique stimulation. Unlike large frog photoreceptors (rod: ˜5 to 8 μm, cone: ˜1 to 3 μm), mouse photoreceptors (1 to 2 μm for both rods and cones) are relatively small. Although individual mouse photoreceptors (FIG. 15A) were not as clear as frog photoreceptors (FIG. 14A), we selected representative individual mouse photoreceptors (arrows 1506 in FIG. 15A), which could be unambiguously isolated from others. FIG. 15B shows temporal displacements of ten mouse photoreceptors pointed out in FIG. 15A. These ten photoreceptors shifted to the left as shown by the arrows in circles 1506 in FIG. 15B. FIG. 15C shows an average magnitude of photoreceptor displacements. As shown in FIG. 15C, the displacement occurred within 5 ms and reached the peak at 30 ms.

Accordingly, high-spatial and temporal-resolution imaging revealed rod-dominant transient phototropic response in frog (FIG. 13) and mouse (FIG. 14) retinas under oblique stimuli. Such transient phototropic response could compensate for the loss of illumination efficiency under oblique stimulation in the rod system.

It is speculated that the observed displacement was rod dominated due to the established knowledge that rods account for ˜97% of total number of the photoreceptors in mouse retinas. In contrast to rods, it can take a long time, at least tens of seconds or even days, for cone adaptation. In other words, rapid (onset: ˜10 ms for frog and ˜5 ms for mouse; time-to-peak: ˜200 ms for frog and ˜20 ms for mouse) phototropic adaptation in retinal rods is too quick for the SCE to be detected by conventional psychophysical methods with the advanced involvement of brain perception. Gaussian-shape stimulation further confirmed the transient rod displacement (FIG. 14). In addition, the observed off-center and on-surround pattern (FIG. 14B) may imply early involvement of the photoreceptors in contrast enhancement. The edge enhancement was confirmed by the IOS maps (FIG. 14C). In general, it is believed that the center-surround antagonism, which is valuable for contrast perception, is initiated by horizontal cells and/or amacrine cells. However, our experimental results here suggest that the discrepancy of the incident angle between the surround and the center of the Gaussian illumination (FIG. 12B) can evoke directional displacement only at the surround (FIG. 14B). Such an edge-enhanced pattern of photoreceptor activity may suggest an early involvement of the photoreceptors in contrast perception.

Moreover, the observed transient rod movement provides an IOS biomarker to allow early detection of eye diseases that can cause retinal dysfunction. Rod function has been well established to be more vulnerable than cones in aging and early AMD, which is the most common cause of severe vision loss and legal blindness in adults over 50. Structural biomarkers, such as drusen and pigmentary abnormalities in the macula, are important for retinal evaluation. Adaptive optics imaging of individual rods has been recently demonstrated. However, the most commonly used tool for retinal imaging, the fundus examination, is not sufficient for a final retinal diagnosis. In principle, physiological function is degraded in diseased cells before detectable abnormality of retinal morphology.

Psychophysical methods and electroretinography measurements have been explored for functional assessment of the retina, but reliable identification of localized rod dysfunctions is still challenging due to limited resolution and sensitivity. The results shown in FIG. 14 indicate that the transient phototropic changes can partially contribute to IOS recording, which has the potential to be developed into a superior non-invasive method for spatiotemporal mapping of retinal function. The different time courses of the IOSs at Zone 1 (periphery) and Zone 2 (center) suggest that the phototropic change of rod photoreceptors primarily contributes to the periphery IOS response. Multiple IOS origins, including neurotransmitter secretion, refractive index change of neural tissues, interactions between photoexcited rhodopsin and GTP-binding protein, disc shape change, cell swelling, etc., have been proposed. In order to investigate the biophysical mechanism of transient phototropic adaptation, we are currently pursuing optical coherence tomography of retinal photoreceptors to quantify the axial location of phototropic kinetics. Further investigations are also planned to quantify time courses of the transient phototropic adaptations in wild type and diseased mouse retinas.

It is anticipated that further investigation of the rod-dominant phototropic effect can provide a high-resolution methodology to achieve objective identification of rod dysfunction, thereby allowing early detection and easy treatment evaluation of eye diseases, such as AMD-associated photoreceptor degeneration.

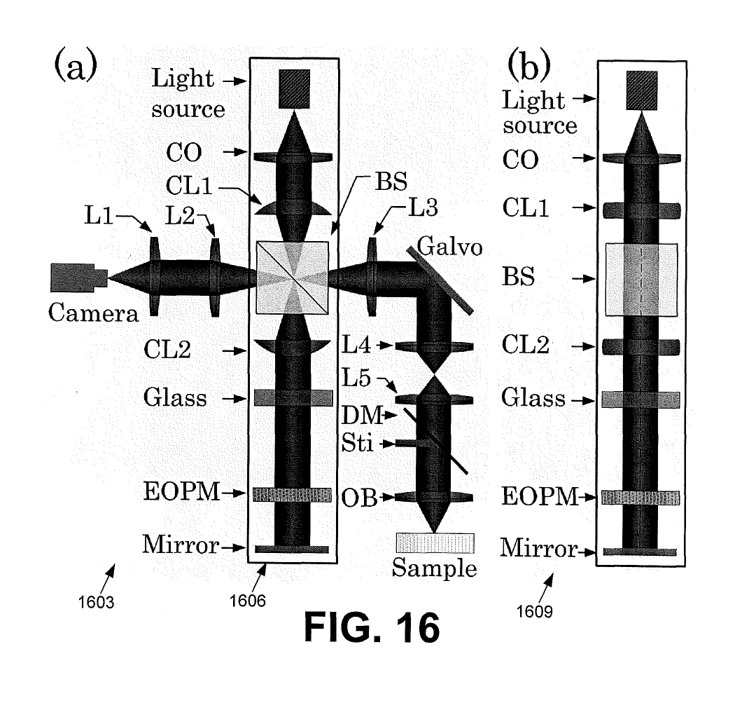

Turning now to FIG. 16, shown is a schematic diagram of a time domain LS-OCT according to various embodiments of the present disclosure. Eyecups of leopard frogs (Rana pipiens) were selected, dark adapted for ˜2 hours, and then euthanized by rapid decapitation and double pithing. Eye balls may then be dissected and moved to Ringer's solution (containing in mM/L: 110 NaCl, 2.5 KCl, 1.6 MgCl2, 1.0 CaCl2, 22 NaHCO3, and 10 D-glucose). An eyecup is made by hemisecting the eye globe below the equator with fine scissors or like device and then removing the lens. Surgical operation may be conducted in a dark room illuminated with dim red light. The eyecup may be immersed in Ringer's solution during functional IOS imaging of the retinal response.

In order to conduct sub-cellular resolution enface IOS imaging of the retina, a rapid time domain line-scan OCT (LS-OCT) system, shown in FIG. 16, may be employed. A LS-OCT may combine technical merits of electro-optic phase modulator (EOPM) modulation and line-scan strategy to achieve rapid, vibration-free OCT imaging.

FIG. 16 shows a schematic diagram of a time domain LS-OCT system 1603. Portion (a) of FIG. 16 shows a top view of the LS-OCT system 1603. According to various embodiments, the LS-OCT system 1603 may comprise a collimator (CO); one or more lenses (L1-L5), with focal lengths of about 80 mm, 40 mm, 80 mm, 40 mm, 75 mm, respectively; an objective (OB) (10×, NA=0.3); one or more cylindrical lenses (CL1 and CL2), with focal lengths of about 75 mm; a beam splitter (BS); a dichroic mirror (DM); a green light stimulus (STI); an electro-optic phase modulator (EOPM), and/or other components. Portion (b) of FIG. 16 depicts a side view 1609 of a rectangle area 1606 in portion (a) of FIG. 16.

A NIR superluminescent diode (SLD-351, Superlum), with a center wavelength of about λ=400 nm to 1000 nm or, in some embodiments, about λ=830 nm and a bandwidth of about Δλ=60 nm, may be used for dynamic OCT imaging. In the illumination path, a cylindrical lens (CL1) may be used to condense the NIR light in one dimension to produce a focused line illumination at the retina. The focused line illumination may be scanned over the retina by a galvo (GVS001, Thorlabs) to achieve rapid enface imaging.

In the reference path, a cylindrical lens (CL2) may be used to convert the focused light back to collimated light. The glass block may be used to compensate for optical dispersion. The EOPM (Model 350-50, Conoptics) may be used to implement vibration-free phase modulation. Light reflected by the mirror and the retina interfered, and is captured by the line-scan camera (Sprint spl2048-140 km, Basler) to retrieve OCT images. The line-scan camera may have a line speed up to about 140,000 lines/s when working at double line mode and about 70,000 lines/s at single line mode. A single line mode may be selected to ensure high resolution of IOS recording. In coordination with the NIR line illumination, the one dimensional CMOS array (1×2048 pixels, 10×10 μm2) of the line-scan camera acts as a slit to achieve a confocal configuration for effective rejection of out-of-focus light.

Using a 10× (NA=0.3) water immersion objective, lateral and axial resolutions of the system were ˜2 μm, (0.61λ/NA), and ˜4 μm (0.44λ2/nΔλ, where n was refractive index of retinal tissue, n≈1.4), respectively.

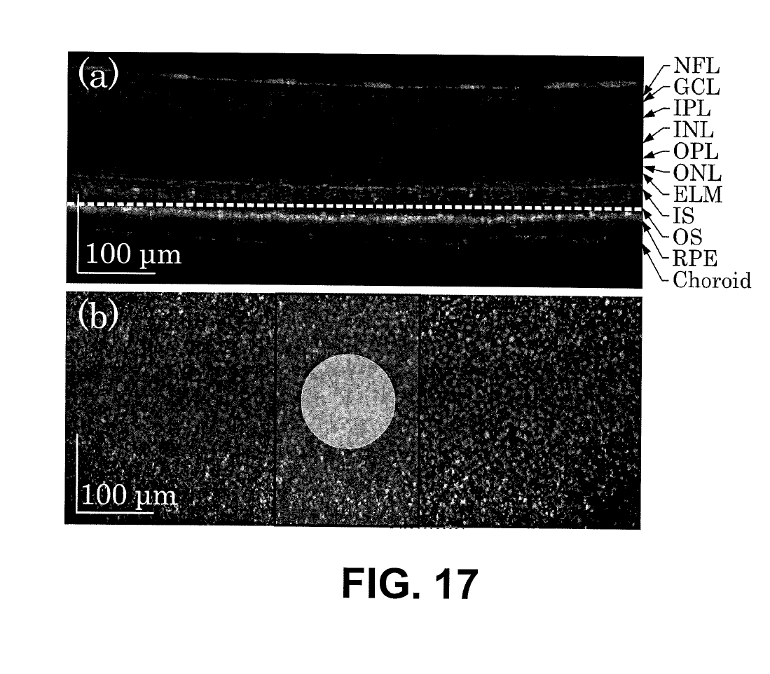

FIG. 17 shows representative time domain LS-OCT images of living frog eyecups obtained according to various embodiments of the present disclosure. The B-scan OCT (portion (a) of FIG. 17), reveals a cross-sectional image of the eyecup that may be reconstructed from a stack of enface OCT images acquired at 50 frames per second (fps). As shown in portion (a) of FIG. 17 clear structures of outer segment (OS), inner segment (IS) ellipsoid, eternal limiting membrane (ELM), outer plexiform layer (OPL), inner nuclear layer (INL), inner plexiform layer (IPL), ganglion cell layer (GCL), and nerve fiber layer (NFL) are shown. In the enface OCT image (portion (b) of FIG. 17), individual photoreceptors could be unambiguously identified.

The OCT recording may be focused at photoreceptor outer segments. For better temporal resolution, the field of view may be reduced and frame speed from 50 fps to 200 fps may be increased. IOS images are presented illustrated with a unit of ΔI/I, where I is the background obtained by averaging pre-stimulus images, and ΔI is the difference between each image and the background. Positive and negative signals were defined by the 3-δ rule.

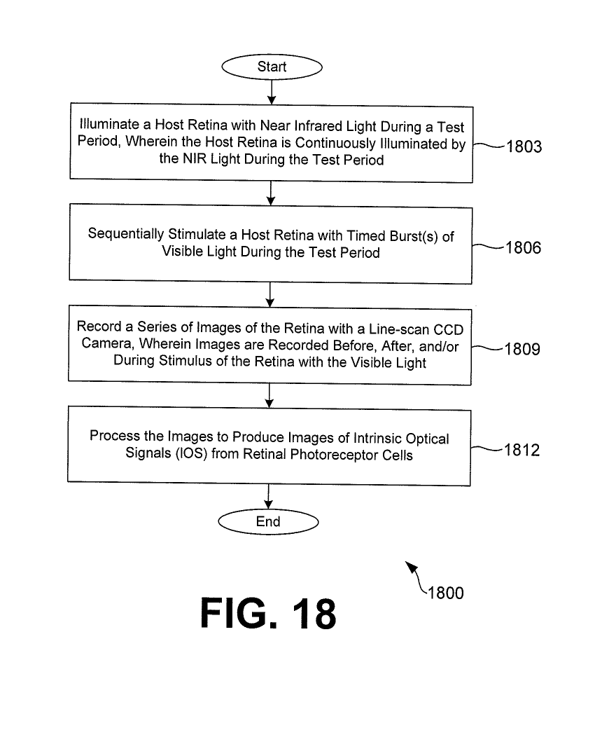

Moving on to FIG. 18, shown is a flowchart 1800 that provides one example of the operation of a portion of imaging retinal intrinsic optical signals (IOS) in vivo according to various embodiments. It is understood that the flowchart of FIG. 18 provides merely an example of the many different types of functional arrangements that may be employed to implement the operation of the portion of imaging retinal intrinsic optical signals (IOS) in vivo as described herein. As an alternative, the flowchart of FIG. 18 may be viewed as depicting an example of elements of a method implemented in a computing environment according to one or more embodiments.

The method may be summarized as: illuminating a host retina with near infrared light during a test period, wherein the host retina is continuously illuminated by an NIR light during the test period (1803); sequentially stimulating the host retina with timed burst(s) of visible light during the test period (1806); recording a series of images of the retina with a line-scan CCD camera, wherein images are recorded before, after, and/or during stimulus of the retina with the visible light (1809); and processing the images to produce images of intrinsic optical signals (IOS) from retinal photoreceptor cells (1812).

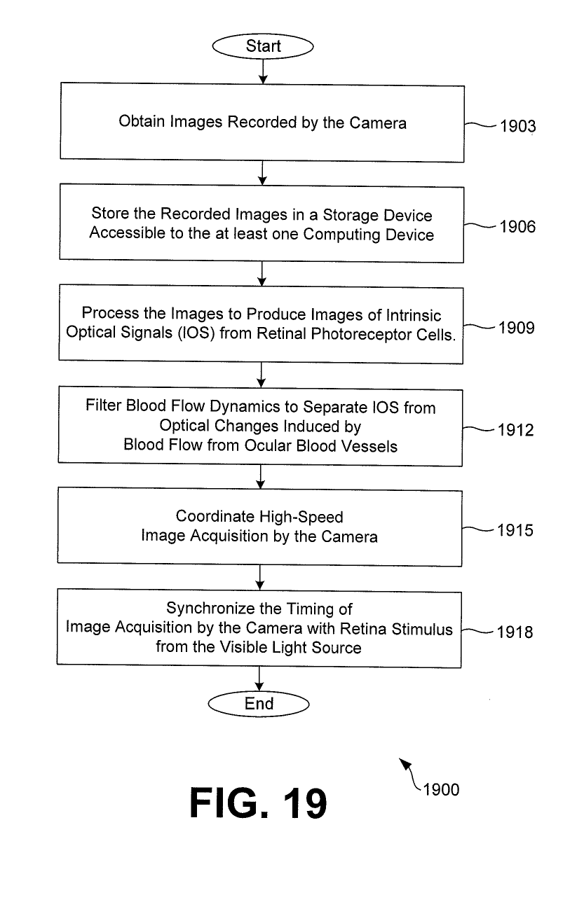

Turning now to FIG. 19, shown is a flowchart 1900 that provides one example of the operation of a portion of imaging retinal intrinsic optical signals (IOS) in vivo according to various embodiments. It is understood that the flowchart of FIG. 19 provides merely an example of the many different types of functional arrangements that may be employed to implement the operation of the portion of imaging retinal intrinsic optical signals (IOS) in vivo as described herein. As an alternative, the flowchart of FIG. 19 may be viewed as depicting an example of elements of a method implemented in a computing environment according to one or more embodiments.

The method may be summarized as: obtaining images recorded by the camera (1903); storing the recorded images in a storage device accessible to the at least one computing device (1906); processing the images to produce images of intrinsic optical signals (IOS) from retinal photoreceptor cells (1909); filtering blood flow dynamics to separate IOS from optical changes induced by blood flow from ocular blood vessels (1912); coordinating high-speed image acquisition by the camera (1915); and synchronizing the timing of image acquisition by the camera with retina stimulus from the visible light source (1918).

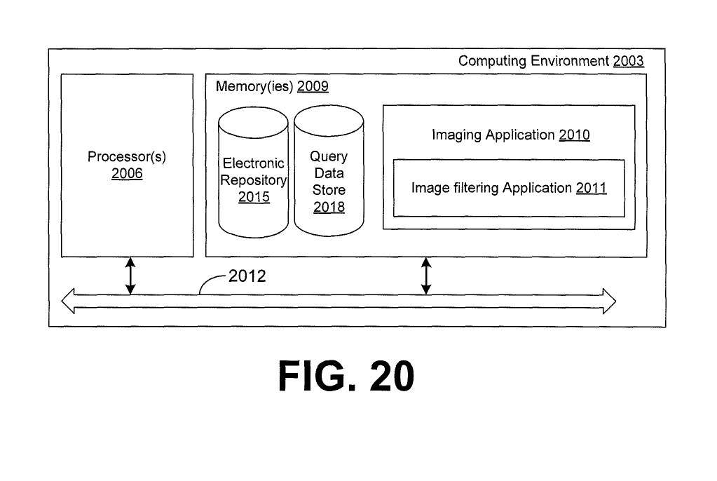

With reference to FIG. 20, shown is a schematic block diagram of a computing environment 2003 according to an embodiment of the present disclosure. The computing environment 2003 includes one or more computing devices, wherein each computing device includes at least one processor circuit, for example, having a processor 2006 and a memory 2009, both of which are coupled to a local interface 2012. To this end, each computing device may comprise, for example, at least one server computer or like device. The local interface 2012 may comprise, for example, a data bus with an accompanying address/control bus or other bus structure as can be appreciated.

Stored in the memory 2009 are both data and several components that are executable by the processor 2006. In particular, stored in the memory 2009 and executable by the processor 2006 are an imaging application 2010 and an image filtering application 2011, and potentially other applications. Also stored in the memory 2009 may be an electronic repository 2015 and a query data store 2018 as well as other data. In addition, an operating system may be stored in the memory 2009 and executable by the processor 2006.

It is understood that there may be other applications that are stored in the memory 2009 and are executable by the processor 2006 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 2009 and are executable by the processor 2006. In this respect, the term “executable” means a program file that is in a form that can ultimately be run by the processor 2006. Examples of executable programs may be, for example, a compiled program that can be translated into machine code in a format that can be loaded into a random access portion of the memory 2009 and run by the processor 2006, source code that may be expressed in proper format such as object code that is capable of being loaded into a random access portion of the memory 2009 and executed by the processor 2006, or source code that may be interpreted by another executable program to generate instructions in a random access portion of the memory 2009 to be executed by the processor 2006, etc. An executable program may be stored in any portion or component of the memory 2009 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 2009 is defined herein as including both volatile and nonvolatile memory and data storage components. Volatile components are those that do not retain data values upon loss of power. Nonvolatile components are those that retain data upon a loss of power. Thus, the memory 2009 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 2006 may represent multiple processors 2006 and/or multiple processor cores and the memory 2009 may represent multiple memories 2009 that operate in parallel processing circuits, respectively. In such a case, the local interface 2012 may be an appropriate network that facilitates communication between any two of the multiple processors 2006, between any processor 2006 and any of the memories 2009, or between any two of the memories 2009, etc. The local interface 2012 may comprise additional systems designed to coordinate this communication, including, for example, performing load balancing. The processor 2006 may be of electrical or of some other available construction.

Although the imaging application 2010 and the image filtering application 2011, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowcharts of FIGS. 18 and 19 show the functionality and operation of an implementation of portions of the imaging application 2010 and/or the image filtering application 2011. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 2006 in a computer system or other system. The machine code may be converted from the source code, etc. If embodied in hardware, each block may represent a circuit or a number of interconnected circuits to implement the specified logical function(s).

Although the flowcharts of FIGS. 18 and 19 show a specific order of execution, it is understood that the order of execution may differ from that which is depicted. For example, the order of execution of two or more blocks may be scrambled relative to the order shown. Also, two or more blocks shown in succession in FIGS. 18 and 19 may be executed concurrently or with partial concurrence. Further, in some embodiments, one or more of the blocks shown in FIGS. 18 and 19 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the imaging application 2010 and the image filtering application 2011, that comprises software or code can be embodied in any non-transitory computer-readable medium for use by or in connection with an instruction execution system such as, for example, a processor 2006 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the imaging application 2010 and the image filtering application 2011, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing environment 2003, or in multiple computing devices in the same computing environment 103. Additionally, it is understood that terms such as “application,” “service,” “system,” “engine,” “module,” and so on may be interchangeable and are not intended to be limiting.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The present disclosure also includes a system for imaging retinal IOS in vivo including a means for obtaining confocal digital images of a host retina; a means for illuminating a host retina with infrared light; a means for stimulating a host retina with visible light; a means for adjusting the area of the retina exposed to the visible light; a means for filtering visible light from the camera; and a means for storing and processing the images recorded by the camera to produce images of retinal IOS.

Methods of the present disclosure also include methods for imaging and/or diagnosing a retinal condition. Briefly described, such methods include imaging a host retina with the imaging system of the present disclosure and obtaining IOS images of the host retinas from the imaging system to determine an IOS distribution pattern for the host retina, where an area of reduced signal in the pattern indicates an area of photoreceptor damage. In embodiments the retinal condition is a retinal injury and/or an outer retinal disease, such as, but not limited to, age-related macular degeneration, retinitis pigmentosa, glaucoma, and diabetic retinopathy.

The present disclosure also includes methods for imaging transient directional change of retinal rods in a host retina including imaging a host retina as described above, where the visible light source is directed at an oblique illumination angle in an illumination area relative to the normal axis of retinal surface or where the visible light is directed in a circular stimulus pattern in an illumination area. The IOS images of host retinas obtained from the imaging system illustrate phototropic displacement of rods in the illumination area. Retinal rod dysfunction can also be imaged by such methods, where an area in the IOS images showing an absence of phototropic rod displacement indicates rod dysfunction.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following embodiments.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value.

1. A method of imaging retinal intrinsic optical signals (IOS) in vivo comprising:
illuminating a host retina with a near infrared light during a test period, wherein the host retina is continuously illuminated by the near infrared light during the test period;
sequentially stimulating a host retina with a timed burst of visible light during the test period;
recording a series of images of the retina with a camera, wherein images are recorded both before, after, and during stimulus of the retina with the visible light; and
processing the images to produce images of intrinsic optical signals (IOS) from retinal photoreceptor cells identified in the images.

2. The method of claim 1, wherein the retina is illuminated with the near infrared light at about 600 μW.

3. The method of claim 1, wherein the visible light is a visible green light.

4. The method of claim 1, wherein the camera further comprises a line-scan CCD camera.

5. The method of claim 4, further comprising filtering the visible light from the line-scan CCD camera with a NIR filter.

6.-7. (canceled)

8. The method of claim 1, wherein the images are recorded at a speed of about 100 frames/s.

9. (canceled)

10. The method of claim 8, wherein the images are recorded for a period of time beginning about 400 ms before the stimulus and continuing until about 800 ms after the stimulus at intervals of about 100 frames/s.

11. (canceled)

12. The method of claim 1, further comprising detecting a reduced IOS signal in an area of an image, wherein the area comprising the reduced IOS signal indicates a location of an injured photoreceptor.

13. The method of claim 1, further comprising obtaining two or more images during each interval and averaging the images for each interval.

14. The method of claim 1, further comprising filtering blood flow dynamics from the image to separate IOS from optical changes induced by blood flow from ocular blood vessels.

15. The method of claim 1, wherein the visible light comprises a white light and wherein the bursts are directed at an oblique angle of about 30° relative to a normal axis of a retinal surface.

16. An imaging system for in vivo retinal imaging of a host retina comprising:
at least one computing device; and
a line-scan confocal ophthalmoscope comprising:
a linear CCD camera;
a near infrared (NIR) light source;
a visible light source;
a scanning mirror;
an adjustable mechanical slit disposed between the visible light source and the host retina; and
a near infrared (NIR) filter disposed between the visible light source and the camera to block visible stimulus light; and
an application executable by the at least one computing device, the application comprising:
logic that obtains images recorded by the camera;
logic that stores the recorded images in a storage device accessible to the at least one computing device; and
logic that processes the images to produce images of intrinsic optical signals (IOS) from retinal photoreceptor cells.

17. The system of claim 16, wherein the application further comprises logic that filters blood flow dynamics to separate IOS from optical changes induced by blood flow from ocular blood vessels.

18. The system of claim 16, wherein application further comprises logic that coordinates a high-speed image acquisition by the camera and synchronizes a timing of image acquisition by the camera with retina stimulus from the visible light source.

19. The system of claim 16, wherein the near infrared light comprises a superluminescent laser diode (SLD).

20.-22. (canceled)

23. The system of claim 16, wherein the visible light source is directed at an oblique illumination angle relative to a normal axis of retinal surface.

24. The system of claim 25, wherein the visible light source is a white light having a wavelength of about 450-650 nm.

25. An imaging system for in vivo retinal imaging of a host retina, comprising:
a line-scan confocal ophthalmoscope comprising:
a linear CCD camera;
a near infrared (NIR) light source;
a visible light source;
a scanning mirror;
an adjustable mechanical slit disposed between the visible light source and the host retina; and
a near infrared (NIR) filter disposed between the visible light source and the camera to block visible stimulus light,
wherein, the system is capable of processing a plurality of images recorded by the camera to produce images of intrinsic optical signals (IOS) from retinal photoreceptor cells.

26. The imaging system of claim 25, wherein the system is capable of filtering blood flow dynamics to separate IOS from optical changes induced by blood flow from ocular blood vessels.

27. The imaging system of claim 25, wherein the images of IOS can indicate injury to retinal photoreceptors.
